(19) United States
(12) Patent Application Publication
(10) Pub. No.: US 2026/0207509 A1
(43) Pub. Date: Jul. 23, 2026

(54) LYOSPHERES CONTAINING PROGRAMMED DEATH RECEPTOR 1 (PD-1) ANTIBODIES AND METHODS OF USE THEREOF

(71) Applicant: MERCK SHARP & DOHME LLC, Rahway, NJ (US)

(72) Inventors: Akhilesh Bhambhani, Blue Bell, PA (US); Smeet Deshmukh, Westfield, NJ (US); Rui Fang, Green Brook, NJ (US); Yogita Krishnamachari, Scotch Plains, NJ (US); Yuwei Lu, Lake Sherwood, CA (US); Geetha Thiagarajan, Westfield, NJ (US)

(73) Assignee: MERCK SHARP & DOHME LLC, Rahway, NJ (US)

(21) Appl. No.: 19/137,362

(22) PCT Filed: Dec. 11, 2023
(86) PCT No.: PCT/US2023/083281

§ 371 Date: Jun. 10, 2025

Related U.S. Application Data
(60) Provisional application No. 63/432,744, filed on Dec. 15, 2022.

(51) Int. Cl. A61K 9/19; A61K 47/26; C07K 16/28

(52) U.S. Cl. CPC A61K 9/19; A61K 47/26; C07K 16/2818; C07K 2317/24

(57) ABSTRACT

The invention relates to stable formulations of antibodies against human programmed death receptor PD-1, or antigen binding fragments thereof. The invention further provides methods for treating various cancers with stable formulations of the invention. In some embodiments of the methods of the invention, the formulations are administered to a subject by subcutaneous administration.

### FIELD OF THE INVENTION

The invention relates to lyospheres containing high concentrations of antibodies that bind to human programmed death receptor 1 (PD-1) and high concentrations of a stabilizer (disaccharide). These lyospheres, once reconstituted, generate a high concentration of PD-I in solution for injection, e.g., subcutaneous delivery.

### BACKGROUND OF THE INVENTION

Immune checkpoint therapies targeting the programmed death receptor-1 (PD-1) axis have resulted in groundbreaking improvements in clinical response in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54: Topalian et al., J Clin Oncol 2014, 32: 1020-30: Wolchok et al., N Engl J Med 2013, 369: 122-33). The interaction of the PD-1 receptor on T-cells with its ligands, PD-L1 and PD-L2, on tumor and immune infiltrating cells regulates T-cell mediated immune responses and may play a role in immune escape by human tumors (Pardoll D M. Nat Rev Cancer 2012, 12: 252-64). Binding of PD-1 to either of its ligands results in delivery of an inhibitory stimulus to the T cell.

Immune therapies targeting the PD-1 axis include monoclonal antibodies directed to the PD-1 receptor (KEYTRUDA™ (pembrolizumab). Merck Sharp & Dohme LLC, Rahway, NJ, USA; OPDIVO™ (nivolumab), Bristol-Myers Squibb Company. Princeton, NJ, USA, and LIBTAYO™ (cemiplimab), Regeneron Pharmaceuticals, Inc., Tarrytown, NY, USA) and also those that bind to the PD-L1 ligand (MPDL3280A; TECENTRIQ™ (atezolizumab), Genentech, San Francisco, CA, USA; IMFINZI™ (durvalumab), AstraZeneca Pharmaceuticals LP, Wilmington, DE; BAVENCIO™ (avelumab), Merck KGaA, Darmstadt, Germany; JEMPERLI™ (dostarlimab). GlaxoSmithKline Biologics LLC, Philadelphia, PA, USA). Both therapeutic approaches have demonstrated anti-tumor effects in numerous cancer types.

Antibody formulations must be stable during storage to ensure efficacy and consistent dosing, so it is critical that whatever formulation is chosen supports desirable properties, such as appropriate antibody concentrations, clarity and acceptable viscosity, while maintaining drug efficacy over an acceptably long shelf-life under typical storage conditions.

Antibody therapies are routinely administered intravenously or via IM injection, but other routes of administration for antibody therapies may be preferred or warranted. For example, subcutaneous administration may be preferred for a number of reasons including 1) ease of administration; 2) lower likelihood of adverse reaction or injection site pain; and 3) patient preference and compliance. Subcutaneous administration of an antibody typically necessitates a formulation comprising a high antibody concentration to render a low volume subcutaneous administration dose. It is known that formulations with a high concentration of mAb in liquid are inherently less stable.

Therefore, there is a need in the art to provide an antibody formulation image that contains a high concentration of antibody and is stable.

### SUMMARY OF THE INVENTION

Liquid formulations comprising high concentrations of antibodies are susceptible to unfavorable aggregation due to protein-protein interaction. To achieve long-term stability, lower concentration formulations can be lyophilized in solid state and subsequently reconstituted to achieve higher concentration prior to administration to patients. Traditional lyophilization technology (lyophilized cake) has been widely applied to develop drug products, however, drying efficiency is often challenged with traditional lyophilization in a vial. Lyosphere technology and the process to make lyospheres is more efficient due to bulk drying and increased surface area in small bead format. For formulations requiring high concentration of stabilizer for optimal stability, lyosphere technology is capable of drying such formulations with high total solid with improved process efficiency, therefore, enabling a wider formulation space than traditional lyophilization in a vial. Preferred stabilizers are disaccharides such as sucrose or trehalose or a combination of sucrose and trehalose, or other disaccharides. It appears that the ratio of a high concentration of antibody to a high concentration of disaccharide is important as well.

Therefore, to enable low volume sub-cutaneous delivery of antibody to patients safely with optimal stability in a solid state, formulations containing high concentrations of antibody with high concentrations of disaccharide have been identified and can be efficiently formulated and manufactured using lyosphere technology.

The present disclosure provides pre-lyosphere formulations, lyospheres and reconstituted lyosphere formulations with high anti-PD-1 antibody and disaccharide content and methods of making and using the same.

In one aspect, the invention provides a pre-lyosphere formulation comprising: a) at least about 25 mg/mL of an anti-PD-1 antibody or antigen binding fragment thereof; b) at least about 25 mg/mL of a stabilizer (disaccharide); c) a buffer, and d) a surfactant.

In some embodiments, the PD-1 antagonist is selected from the group consisting of: pembrolizumab (KEYTRUDA™, Merck Sharp & Dohme LLC, Rahway, NJ, USA), nivolumab (OPDIVO™, Bristol-Myers Squibb Company, Princeton, NJ, USA), atezolizumab (TECENTRIQ™ Genentech, San Francisco, CA, USA), durvalumab (IMFINZI™, AstraZeneca Pharmaceuticals LP, Wilmington, DE), cemiplimab (LIBTAYO™, Regeneron Pharmaceuticals, Tarrytown, NY, USA) avelumab (BAVENCIO™, Merck KGaA. Darmstadt. Germany), dostarlimab (JEMPERLI™, GlaxoSmithKline LLC, Philadelphia, PA), and pidilizumab (U.S. Pat. No. 7,332,582).

In certain embodiments, the anti-PD-1 antibody is pembrolizumab.

In other embodiments the anti-PD-1 antibody is nivolumab.

In further embodiments the anti-PD-1 antibody is atezolizumab.

In yet other embodiments, the anti-PD-1 antibody is cemiplimab.

In still other embodiments the anti-PD-1 antibody is avelumab.

In specific embodiments the anti-PD-1 antibody is dostarlimab.

In specific embodiments the anti-PD-1 antibody is pidilizumab.

In certain embodiments the stabilizer (disaccharide) is sucrose or trehalose, or a combination of sucrose and trehalose.

In other embodiments, the stabilizer (disaccharide) is a combination of sucrose and trehalose, wherein the concentration of trehalose is greater than the concentration of sucrose.

In other embodiments, the anti-PD-1 antibody is at a concentration of about 25 mg/mL to about 200 mg/mL, or at a concentration of about 50 mg/mL to about 200 mg/mL, or at a concentration of about 100 mg/mL to about 200 mg/mL, or at a concentration of about 150 mg/mL to about 200 mg/mL. In some embodiments, the anti-PD-I antibody is at a concentration of about 225 mg/mL, or about 250 mg/mL, or about 275 mg/mL, or about 300 mg/mL or greater.

In some embodiments, the disaccharide is at a concentration of 25 mg/mL to 250 mg/mL, or at a concentration of 50 mg/mL to 200 mg/mL, or at a concentration of 100 mg/mL to 200 mg/mL, or at a concentration of 150 mg/mL to 200 mg/mL. In some embodiments, the disaccharide is at a concentration of 200 mg/mL to 300 mg/mL. In some embodiments, the disaccharide is at a concentration of about 210 mg/mL, or about 220 mg/mL, or about 230 mg/mL, or about 240 mg/mL, or about 250 mg/mL, or about 260 mg/mL, or about 270 mg/mL, or about 280 mg/mL, or about 290 mg/mL, or about 300 mg/mL, or greater than 300 mg/mL.

In some embodiments, the disaccharide is present in an amount of about 20% weight/volume (w/v), or about 25% w/v, or about 30% w/v.

In some embodiments, the buffer is selected from the group consisting of potassium phosphate, sodium phosphate, sodium acetate, histidine, imidazole, sodium citrate, sodium succinate, ammonium bicarbonate, HEPES, Tris, Bis-Tris, and a carbonate, and/or combinations thereof.

In some embodiments the buffer is at a concentration of about 5-50 mM.

In some embodiments the buffer is at a concentration of about 5-15 mM.

In some embodiments the buffer is at a concentration of about 10-20 mM.

In some embodiments the buffer is at a concentration of about 10 mM.

In some embodiments the buffer is at a concentration of 10 mM.

In some embodiments the buffer is histidine.

In certain embodiments, the surfactant is a non-ionic surfactant.

In some embodiments, the surfactant is polysorbate 80 (PS-80), or polysorbate 20 (PS-20).

In some embodiments the surfactant is PS-80.

In some embodiments the surfactant is PS-20.

In some embodiments the surfactant is present in an amount of about 0.01-0.2% wv/v.

In some embodiments the surfactant is present in an amount of about 0.02-0.04% w/v.

In some embodiments the surfactant is at a concentration of about 0.1-2 mg/mL.

In some embodiments the surfactant is at a concentration of about 0.2-0.4 mg/mL.

In some embodiments the concentration ratio of anti-PD-1 antibody to disaccharide is 1:1.

In some embodiments the pH of the pre-lyosphere formulation is about 5.0 to about 6.5.

In some embodiments the pH of the pre-lyosphere formulation is about 5.5 to about 6.0.

In some embodiments the pre-lyosphere formulation further comprises a viscosity reducer.

In some embodiments the viscosity reducer is arginine, lactic acid, or nicotinic acid and/or combinations thereof.

In one embodiment, the pre-lyosphere formulation comprises: a) about 25 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In another embodiment, the pre-lyosphere formulation comprises: a) about 50 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In one embodiment the pre-lyosphere formulation comprises: a) about 75 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In one embodiment the pre-lyosphere formulation comprises: a) about 100 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In one embodiment the pre-lyosphere formulation comprises: a) about 125 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In one embodiment the pre-lyosphere formulation comprises: a) about 150 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In one embodiment the pre-lyosphere formulation comprises: a) about 175 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04%% w/v PS-80.

In one embodiment the pre-lyosphere formulation comprises: a) about 200 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% N w/v PS-80.

In one embodiment the pre-lyosphere formulation comprises: a) about 225 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In one embodiment the pre-lyosphere formulation comprises: a) about 250 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In one embodiment the pre-lyosphere formulation comprises: a) about 275 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In one embodiment the pre-lyosphere formulation comprises: a) about 300 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In one aspect, the invention provides a lyosphere (50 pl volume/bead) comprising: a) 1.25 mg to 10 mg of an anti-PD-1 antibody; b) 1.25 mg to 12.5 mg of a stabilizer (disaccharide); and c) 0.005 to 0.01 mg of surfactant.

In certain embodiments of the lyosphere of the invention, the anti-PD-1 antibody is pembrolizumab.

In other embodiments the anti-PD-1 antibody is nivolumab.

In further embodiments the anti-PD-1 antibody is atezolizumab.

In yet other embodiments, the anti-PD-1 antibody is cemiplimab.

In still other embodiments the anti-PD-1 antibody is avelumab.

In specific embodiments the anti-PD-1 antibody is dostarlimab.

In specific embodiments the anti-PD-1 antibody is pidilizumab.

In some embodiments, the lyosphere comprises about 1.25 mg, or about 2 mg, or about 4 mg, or about 6 mg, or about 8 mg, or about 10 mg anti-PD-1 antibody.

In some embodiments, the lyosphere comprises about 1.25 mg, or about 2 mg, or about 4 mg, or about 6 mg, or about 8 mg, or about 10 mg, or about 12 mg, or about 12.5 mg stabilizer (disaccharide).

In some embodiments, the stabilizer (disaccharide) is sucrose or trehalose, or a combination of sucrose and trehalose.

In some embodiments, the stabilizer (disaccharide) is a combination of sucrose and trehalose, wherein the concentration of trehalose is greater than concentration of sucrose.

In some embodiments, the surfactant is a non-ionic surfactant.

In some embodiments the surfactant is a polysorbate 80 (PS-80), or a polysorbate 20 (PS-20).

In some embodiments the surfactant is PS-80.

In some embodiments the surfactant is PS-20.

In some embodiments the lyosphere comprises about 0.05 to about 0.1 mg surfactant.

In some embodiments the lyosphere comprises about 0.5 to about I mg surfactant.

In some embodiments the concentration ratio of anti-PD-1 antibody to disaccharide is 1:1.

In another aspect, the lyosphere is reconstituted (a reconstituted lyosphere formulation).

In some embodiments the pH of the reconstituted lyosphere formulation is about 5.0 to about 6.5.

In some embodiments the pH of the reconstituted lyosphere formulation is about 5.5 to about 6.0.

In some embodiments the reconstituted lyosphere formulation further comprises a viscosity reducer.

In some embodiments, the viscosity reducer is arginine, lactic acid, or nicotinic acid and/or combinations thereof.

In some embodiments, the reconstituted lyosphere formulation (to be administered to a patient) comprises: a) 130 mg/mL to 200 mg/mL of the anti-PD-1 antibody; b) 130 mg/mL to 200 mg/mL stabilizer (disaccharide); c) 1 to 20 mM buffer, pH 5.0-6.5; and d) about 0.02-0.04% w/v surfactant.

In some embodiments the anti-PD-1 antibody is pembrolizumab.

In some embodiments the anti-PD-1 antibody is nivolumab.

In further embodiments the anti-PD-1 antibody is atezolizumab.

In yet other embodiments, the anti-PD-1 antibody is cemiplimab.

In still other embodiments the anti-PD-1 antibody is avelumab.

In specific embodiments the anti-PD-1 antibody is dostarlimab.

In specific embodiments the anti-PD-1 antibody is pidilizumab.

In some embodiments the stabilizer (disaccharide) is sucrose or trehalose, or a combination of sucrose and trehalose.

In some embodiments the stabilizer (disaccharide) is a combination of sucrose and trehalose, wherein the concentration of trehalose is greater than concentration of sucrose.

In some embodiments the buffer is selected from the group consisting of potassium phosphate, sodium phosphate, sodium acetate, histidine, imidazole, sodium citrate, sodium succinate, ammonium bicarbonate, HEPES, Tris, Bis-Tris, and a carbonate, and/or combinations thereof.

In some embodiments the buffer is histidine.

In some embodiments the surfactant is a non-ionic surfactant.

In some embodiments the surfactant is polysorbate 80 (PS-80), or polysorbate 20 (PS-20).

In other embodiments the surfactant is PS-80.

In other embodiments the surfactant is PS-20.

In some embodiments the reconstituted lyosphere formulation further comprises a viscosity reducer.

In some embodiments the viscosity reducer is arginine, lactic acid, or nicotinic acid and/or combinations thereof.

In one embodiment, the reconstituted lyosphere formulation (to be administered to a patient) comprises: a) 130 mg/mL to 200 mg/mL of the anti-PD-1 antibody; b) 130 mg/mL to 200 mg/mL trehalose, or about 130 mg/mL to 200 mg/mL sucrose, or a combination of trehalose and sucrose totaling about 130 mg/mL to 200 mg/mL: c) 10 mM histidine buffer, pH 5.5-6.0; and d) about 0.02-0.04% PS80.

In certain embodiments, the reconstituted lyosphere formulation is useful for treating a disease or condition.

In certain embodiments, the reconstituted lyosphere formulation is useful for the treatment of cancer.

In certain embodiments, the reconstituted lyosphere formulation is useful for sub-cutaneous administration.

In another aspect, the present invention provides a method for preparing a lyosphere comprising the steps of: a) preparing a pre-lyosphere formulation as described herein; b) freezing the pre-lyosphere formulation, thereby forming at least one frozen body; and c) drying the frozen body to produce the lyosphere.

In one embodiment of the method, the drying step is performed in a container selected from the group consisting of a tray, a bag, and a vial.

In some embodiments of the method, the lyosphere is prepared via a sublimated drying process.

In some embodiments of the method, the sublimative drying process comprises lyophilization or radiant energy vacuum (REV) dehydration in travelling wave format.

In certain embodiments of the method, the freezing step is performed at ≤−50° C., or at ≤−100° C., or at ≤−150° C. In certain embodiments, the freezing step is flash freezing. In various embodiments, the freezing step is performed between −50° C., and −100° C., or between −100° C., and −150° C.

### BRIEF DESCRIPTION OF THE DRAWINGS

FIG. 1 is a graph showing percentage of aggregates (as measured by high performance size-exclusion chromatography; HP-SEC) as a function of time at 40° C. for anti-PD-1 antibody formulations containing various levels of disaccharide (pre-lyosphere formulations) dried by lyophilization drying.

FIG. 2 is a graph showing percentage of high molecular weight (HMW) aggregates as a function of time at 25° C., and 40° C. for anti-PD-1 antibody lyosphere formulations after reconstitution.

FIG. 3 is a graph showing reconstitution times in seconds (s) as a function of antibody concentration for anti-PD-1 antibody lyospheres.

FIG. 4 is a graph showing reconstitution times in seconds and viscosity for anti-PD-1 antibody lyophilized cakes and anti-PD-1 antibody lyospheres after drying.

FIG. 5 is a graph showing percentage of HMW aggregates in anti-PD-1 (Formulations F1, F2 and F3) antibody lyophilized cakes and lyospheres stored for 12-months at 40° C. at 75% RH or at 25° C. at 60% RH.

FIG. 6 is a graph showing percentage of oxidation in anti-PD-1 (Formulations F1, F2 and F3) antibody formulation (pre-lyosphere formulations), lyophilized cakes and lyophilized cakes stored for 12-months at 40° C., or 25° C.

FIG. 7, is a graph showing percentage of aggregates (as measured by high performance size-exclusion chromatography; HP-SEC) as a function of time at 25° C., and 40° C. for anti-PD-1 antibody formulations at 165 mg/mL with 70 mg/mL of sucrose.

FIG. 8, is a graph showing percentage of charge variants (as measured by IEX) as a function of time at 25° C., and 40° C. for anti-PD-1 antibody formulations at 165 mg/mL with 70 mg/mL of sucrose.

### DETAILED DESCRIPTION OF THE INVENTION

The invention provides a pre-lyosphere formulation, a lyosphere and a reconstituted lyosphere formulation comprising a high concentration of an anti-PD-1 antibody and a high concentration of a disaccharide. In various embodiments, the reconstituted lyosphere formulation is effective for active treatment of diseases or disorders. In various embodiments, the lyosphere is thermostable and suitable for long term storage (e.g., more than 12 months at room temperature with available data). In various embodiments, the reconstituted lyosphere formulation is useful for subcutaneous delivery to a subject, e.g., human subject/patient for treatment of a disease and/or condition.

### Definitions and Abbreviations

As used throughout the specification and appended claims, the following abbreviations apply:
API active pharmaceutical ingredient
CDR complementarity determining region in the immunoglobulin variable regions
CE-SDS capillary electrophoresis-sodium dodecyl sulfate
CHO Chinese hamster ovary
CI confidence interval
DS drug substance
EC50 concentration resulting in 50% efficacy or binding
ELISA enzyme-linked immunosorbant assay
FFPE formalin-fixed, paraffin-embedded
FR framework region
FTIR Fourier Transform Infrared Spectroscopy
HC heavy chain
HNSCC head and neck squamous cell carcinoma
HPBC 2-Hydroxypropyl)-o-cyclodextrin
HP-HIC high performance hydrophobic interaction chromatography
HP-IEX high performance ion-exchange chromatography
HP-SEC high performance size exclusion chromatography
TC50 concentration resulting in 50% inhibition
IgG immunoglobulin G
IHC immunohistochemistry or immunohistochemical
mAb monoclonal antibody
MES 2-(N-morpholino)ethanesulfonic acid
NCBI National Center for Biotechnology Information
NSCLC non-small cell lung cancer
PCR polymerase chain reaction
PD-1 programmed death 1 (a.k.a. programmed cell death-1 and programmed death receptor 1)
PD-L1 programmed cell death 1 ligand 1
PD-L2 programmed cell death 1 ligand 2
PS80 or PS-80 polysorbate 80
RH relative humidity
SBEC (sulfobutylether)-p-cyclodextrin
SWFI sterile water for injection
TNBC triple negative breast cancer
V_{H} immunoglobulin heavy chain variable region
VK immunoglobulin kappa light chain variable region
V_{L} immunoglobulin light chain variable region
VP-DSC Valerian-Plotnikov differential scanning calorimetry
v/v volume per volume
WFI water for injection
w/v weight per volume

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

“Treat” or “treating” a cancer as used herein means to administer a reconstituted lyosphere formulation of the invention to a subject having an immune condition or cancerous condition, or diagnosed with a cancer or pathogenic infection (e.g. viral, bacterial, fungal), to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, stimulating an immune response to a pathogen, toxin, and/or self-antigen, stimulating an immune response to a viral infection, decreasing the number of one or more tumor markers, halting or delaying the growth of a tumor or blood cancer or progression of disease associated with PD-1 binding to its ligands PD-L1 and/or PD-L2 (“PD-I-related disease”) such as cancer, stabilization of PD-I-related disease, inhibiting the growth or survival of tumor cells, eliminating or reducing the size of one or more cancerous lesions or tumors, decreasing the level of one or more tumor markers, ameliorating, abrogating the clinical manifestations of PD-1-related disease, reducing the severity or duration of the clinical symptoms of PD-I-related disease such as cancer, prolonging the survival of a patient relative to the expected survival in a similar untreated patient, inducing complete or partial remission of a cancerous condition or other PD-1 related disease.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

The term “patient” (alternatively referred to as “subject” or “individual” herein) refers to a mammal (e.g., rat, mouse, dog, cat, rabbit) capable of being treated with the reconstituted lyosphere formulation of the invention, most preferably a human. In some embodiments, the patient is an adult patient. In other embodiments, the patient is a pediatric patient. Those “in need of treatment” include those patients that may benefit from treatment with the reconstituted lyosphere formulation of the invention, e.g., a patient suffering from cancer or an immune condition.

The term “antibody” refers to any form of antibody that exhibits the desired biological activity. Thus, it is used in the broadest sense and specifically covers, but is not limited to, monoclonal antibodies (including full length monoclonal antibodies), polyclonal antibodies, humanized, fully human antibodies, and chimeric antibodies. In some embodiments the antibody is an anti-PD-1 antibody or antigen binding fragments thereof. In some embodiments the antibody is an anti-PD-1 antibody. In some embodiments the antibody is pembrolizumab or nivolumab.

The term “pharmaceutically effective amount” or “effective amount” means an amount whereby sufficient therapeutic (in a reconstituted lyosphere formulation) is introduced to a patient to treat a diseased or condition. One skilled in the art recognizes that this level may vary according to the patient's characteristics such as age, weight, etc.

The term “about”, when modifying 1) the quantity (e.g., mM, or M) of a substance, formulation, pre-lyosphere formulation, lyosphere, or reconstituted lyosphere formulation; 2) the percentage (v/v or w/v) of a substance, formulation, pre-lyosphere formulation, lyosphere, or reconstituted lyosphere formulation; 3) the pH of a substance, formulation, pre-lyosphere formulation, lyosphere, or reconstituted lyosphere formulation; or 4) the value of a parameter characterizing a step in a method, or the like, refers to variation in the numerical quantity that can occur, for example, a) through typical measuring, handling and sampling procedures involved in the preparation, characterization and/or use of a substance, formulation, pre-lyosphere formulation, lyosphere, or reconstituted lyosphere formulation: b) through inadvertent error in these procedures; or c) through differences in the manufacture, source, or purity of the ingredients employed to make or use the substances, formulations, pre-lyosphere formulations, lyospheres, or reconstituted lyosphere formulations, or carry out the procedures; and the like; and is meant to capture variation up to and including 10% of the value. In certain embodiments, “about” can mean a variation of ±0.1%, 0.5%, 1%, 2%, 3%, 4%, 5% or 10%.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

As used herein. “x % (w/v)” is equivalent to x g/100 ml (for example 5% w/v equals 50 mg/ml).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

The term “buffer” encompasses those agents which maintain the solution pH of the pre-lyosphere formulations or reconstituted lyosphere formulations of the invention in an acceptable range.

The terms “lyophilization,” “lyophilized,” and “freeze-dried” refer to a process by which material (i.e., material in a pre-lyosphere formulation) to be dried is first frozen and then the ice or frozen solvent is removed by sublimation in a vacuum environment (i.e., a process for making the lyosphere).

The term “pharmaceutical formulation” refers to preparations which are in such form as to permit the active ingredients (antibodies) to be effective, and which contains no additional components which are toxic to the subjects to which the formulation would be administered. The term “pharmaceutical formulation” and “reconstituted lyosphere formulation” are used interchangeably throughout.

“Pharmaceutically acceptable” refers to excipients (vehicles, additives) and pharmaceutical formulations that can reasonably be administered to a subject to provide an effective dose of the active ingredient (antibody, for example pembrolizumab) employed and that are “generally regarded as safe” e.g., that are physiologically tolerable and do not typically produce an allergic or similar untoward reaction, such as gastric upset and the like, when administered to a human. In another embodiment, this term refers to molecular entities (antibodies, for example anti-PD-I antibodies) and pharmaceutical formulations approved by a regulatory agency of the federal or a state government or listed in the U.S. Pharmacopeia or another generally recognized pharmacopeia for use in animals, and more particularly in humans. A reconstituted lyosphere formulation of the instant invention is a pharmaceutical formulation and is a pharmaceutically acceptable formulation.

A “reconstituted lyosphere formulation” is a pharmaceutical formulation that has been prepared by dissolving a lyosphere in a diluent such that the antibody (for example pembrolizumab) is dispersed in the pharmaceutical formulation. The reconstituted lyosphere formulation is suitable for administration, e.g., subcutaneous administration).

“Reconstitution time” is the time that is required to rehydrate a lyosphere with a solution to a particle-free clarified solution (i.e., the reconstituted lyosphere formulation or pharmaceutical formulation).

A “stable” lyosphere or a “stable” reconstituted lyosphere formulation is an image or article of manufacture or pharmaceutical formulation in which the antibody (for example pembrolizumab) therein essentially retains its physical stability and/or chemical stability and/or biological activity upon storage. Various analytical techniques for measuring antibody stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. Stability can be measured at a selected temperature for a selected time period. For example, in one embodiment, a stable lyosphere or reconstituted lyosphere formulation is an image or article of manufacture or pharmaceutical formulation with no significant changes observed at a refrigerated temperature (2-8° C.) for at least 12 months. In another embodiment, a stable lyosphere or reconstituted lyosphere formulation is an image or article of manufacture or pharmaceutical formulation with no significant changes observed at a refrigerated temperature (2-8° C.) for at least 18 months. In another embodiment, a stable lyosphere or reconstituted lyosphere formulation is an image or article of manufacture or pharmaceutical formulation with no significant changes observed at room temperature (23-27° C.) for at least 3 months. In another embodiment, a stable lyosphere or reconstituted lyosphere formulation is an image or article of manufacture or pharmaceutical formulation with no significant changes observed at room temperature (23-27° C.) for at least 6 months. In another embodiment, a stable lyosphere or reconstituted lyosphere formulation is an image or article of manufacture or pharmaceutical formulation with no significant changes observed at room temperature (23-27° C.) for at least 12 months. In another embodiment, a stable lyosphere or reconstituted lyosphere formulation is an image or article of manufacture or pharmaceutical formulation with no significant changes observed at room temperature (23-27° C.) for at least 18 months. The criteria for stability for a lyosphere or a reconstituted lyosphere formulation are as follows below. Typically, no more than 10%, preferably 5%, of antibody monomer is degraded as measured by SEC-HPLC. Typically, the reconstituted lyosphere formulation is colorless, or clear to slightly opalescent by visual analysis. Typically, the concentration, pH and osmolality of the lyosphere or reconstituted lyosphere formulation has no more than +/−10% change. Potency is typically within 60-140%, preferably 80-120% of the control or reference. Typically, no more than 10%, preferably 5% of clipping of the antibody is observed, i.e., % low molecular weight species as determined, for example, by HP-SEC. Typically, no more than 10%, preferably no more than 5% of aggregation of the antibody is observed, i.e., % high molecular weight species as determined, for example, by HP-SEC. The term “thermostable lyosphere” or “thermostable reconstituted lyosphere formulation) refers to a lyosphere or reconstituted lyosphere formulation that is not readily destroyed or deactivated over a period of time by room temperature or elevated (i.e., above room temperature) heat. The lyospheres and reconstituted lyosphere formulations of the instant invention are stable.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering. The changes of antibody conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the antibody. Degradation processes that often alter the antibody chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding assay. Pharmaceutical formulations of the invention include antibodies that are biologically active when reconstituted or in liquid form.

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

A “non-reducing sugar” is a sugar not capable of acting as a reducing agent because it does not contain or cannot be converted to contain a free aldehyde group or a free ketone group. Examples of non-reducing sugars include but are not limited to disaccharides such as sucrose and trehalose.

“Pembrolizumab” (formerly known as MK-3475, SCH 900475 and lambrolizumab) is a humanized IgG4 mAb with the structure described in WHO Drug Information, Vol. 27, No. 2, pages 161-162 (2013) and which comprises the heavy and light chain amino acid sequences and CDRs described in Table 1. Pembrolizumab has been approved by the U.S. FDA for the treatment of patients with unresectable or metastatic melanoma, for the adjuvant treatment of certain patients with melanoma, for the treatment of certain patients with recurrent or metastatic head and neck squamous cell cancer (HNSCC), classical Hodgkin lymphoma (cHL), primary mediastinal large B-cell lymphoma, urothelial carcinoma, gastric cancer, microsatellite instability-high (MSI-H) cancer, non-small cell lung cancer, esophageal cancer, cervical cancer, hepatocellular carcinoma, renal cell carcinoma. Merkel cell carcinoma, endometrial carcinoma, cutaneous squamous cell carcinoma, triple negative breast cancer and for the treatment of tumor mutational burden-high cancer, as described in the Prescribing Information for KEYTRUDA™ (Merck & Co., Inc., Rahway, NJ USA; initial U.S. approval 2014, updated August 2022).

As used herein, an “antibody variant” e.g., a “pembrolizumab variant” means a monoclonal antibody which comprises heavy chain and light chain sequences that are identical to those of the reference antibody, e.g., pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

### Formulations of the Invention

### (a) Pre-Lyosphere Formulation

The invention provides a pre-lyosphere formulation comprising: a) at least about 25 mg/mL of an anti-PD-1 antibody; b) at least about 25 mg/mL of a stabilizer (disaccharide): c) a buffer, and d) a surfactant.

In certain embodiments, the anti-PD-1 antibody is pembrolizumab.

In some embodiments, the anti-PD-1 antibody is nivolumab.

In some embodiments, the stabilizer (disaccharide) is sucrose or trehalose, or a combination of sucrose and trehalose.

In some embodiments, the stabilizer (disaccharide) is a combination of sucrose and trehalose, wherein the concentration of trehalose is greater than concentration of sucrose.

In some embodiments, the anti-PD-1 antibody is at a concentration of 25 mg/mL to 200 mg/mL, or at a concentration of 50 mg/mL to 200 mg/mL, or at a concentration of 100 mg/mL to 200 mg/mL, or at a concentration of 150 mg/mL to 200 mg/mL. In some embodiments the anti-PD-1 antibody is at a concentration of about 225 mg/mL, or about 250 mg/mL, or about 275 mg/mL, or about 300 mg/mL or greater.

In another embodiment the disaccharide is at a concentration of 25 mg/mL to 250 mg/mL, or at a concentration of 50 mg/mL to 200 mg/mL, or at a concentration of 100 mg/mL to 200 mg/mL, or at a concentration of 150 mg/mL to 200 mg/mL. In some embodiments the disaccharide is at a concentration of 200 mg/mL to 300 mg/mL. In some embodiments the disaccharide is at a concentration of about 210 mg/mL, or about 220 mg/mL, or about 230 mg/mL, or about 240 mg/mL, or about 250 mg/mL, or about 260 mg/mL, or about 270 mg/mL, or about 280 mg/mL, or about 290 mg/mL, or about 300 mg/mL.

In another embodiment the disaccharide is about 20% weight/volume (w/v), or about 25% w/v, or about 30% w/v.

In another embodiment, the buffer is selected from the group consisting of potassium phosphate, sodium phosphate, sodium acetate, histidine, imidazole, sodium citrate, sodium succinate, ammonium bicarbonate, HEPES. Tris, Bis-Tris, and a carbonate, and/or combinations thereof.

In another embodiment, the buffer is at a concentration of about 5-50 mM.

In another embodiment, the buffer is at a concentration of about 5-15 mM.

In another embodiment, the buffer is at a concentration of about 10-20 mM.

In another embodiment, the buffer is at a concentration of about 10 mM.

In another embodiment, the buffer is at a concentration of 10 mM.

In another embodiment, the buffer is histidine.

In another embodiment, the surfactant is a non-ionic surfactant.

In another embodiment, the surfactant is polysorbate 80 (PS-80), or polysorbate 20 (PS-20).

In another embodiment, the surfactant is PS-80.

In another embodiment, the surfactant is PS-20.

In another embodiment, the surfactant is at a concentration of about 0.01-0.2%.

In another embodiment, the surfactant is at a concentration of about 0.02-0.04%.

In another embodiment, the surfactant is at a concentration of about 0.1-2 mg/mL.

In another embodiment, the surfactant is at a concentration of about 0.2-0.4 mg/mL.

In another embodiment, the concentration ratio of anti-PD-1 antibody to disaccharide is 1:1.

In another embodiment, the pH of the pre-lyosphere formulation is about 5.0 to about 6.5.

In another embodiment, the pH of the pre-lyosphere formulation is about 5.5 to about 6.0.

In another embodiment, the pre-lyosphere formulation further comprises a viscosity reducer.

In another embodiment, the viscosity reducer is arginine, lactic acid, or nicotinic acid and/or combinations thereof.

In another embodiment, the pre-lyosphere formulation comprises: a) about 25 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In another embodiment the pre-lyosphere formulation comprises: a) about 50 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In another embodiment, the pre-lyosphere formulation comprises: a) about 75 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In another embodiment, the pre-lyosphere formulation comprises: a) about 100 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In another embodiment, the pre-lyosphere formulation comprises: a) about 125 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In another embodiment the pre-lyosphere formulation comprises: a) about 150 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In another embodiment, the pre-lyosphere formulation comprises: a) about 175 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In another embodiment, the pre-lyosphere formulation comprises: a) about 200 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% N w/v PS-80.

In another embodiment, the pre-lyosphere formulation comprises: a) about 225 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% N w/v PS-80.

In another embodiment the pre-lyosphere formulation comprises: a) about 250 mg/mL of pembrolizumab; b) about 250 mg/mL trehalose, or about 250 mg/mL sucrose, or a combination of trehalose and sucrose totaling about 250 mg/mL; c) about 10 mM histidine buffer. pH 5.5-6.0; and d) about 0.02-0.04% w/v PS-80.

In another embodiment the pre-lyosphere formulation comprises: a) about 275 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

In another embodiment the pre-lyosphere formulation comprises: a) about 300 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% w/v PS-80.

### (b) Lyospheres

The invention further provides a lyosphere (50 pl volume/bead) comprising: a) 1.25 mg to 10 mg of an anti-PD-1 antibody; b) 1.25 mg to 12.5 mg of a stabilizer (disaccharide); and c) 0.005 to 0.01 mg of surfactant.

In one embodiment the anti-PD-1 antibody is pembrolizumab.

In one embodiment the anti-PD-1 antibody is nivolumab.

In a further embodiment the anti-PD-1 antibody is atezolizumab.

In yet another embodiment, the anti-PD-1 antibody is cemiplimab.

In still another embodiment the anti-PD-1 antibody is avelumab.

In a specific embodiment the anti-PD-1 antibody is dostarlimab.

In another embodiment the anti-PD-1 antibody is pidilizumab.

In one embodiment the anti-PD-1 antibody is at a concentration of about 1.25 mg, or about 2 mg, or about 4 mg, or about 6 mg, or about 8 mg, or about 10 mg.

In one embodiment, the stabilizer (disaccharide) is at a concentration of about 1.25 mg, or about 2 mg, or about 4 mg, or about 6 mg, or about 8 mg, or about 10 mg, or about 12 mg, or about 12.5 mg.

In another embodiment the stabilizer (disaccharide) is sucrose or trehalose, or a combination of sucrose and trehalose.

In another embodiment the stabilizer (disaccharide) is a combination of sucrose and trehalose, wherein the concentration of trehalose is greater than concentration of sucrose.

In another embodiment the surfactant is a non-ionic surfactant.

In another embodiment the surfactant is polysorbate 80 (PS-80), or polysorbate 20 (PS-20).

In another embodiment the surfactant is PS-80.

In another embodiment the surfactant is PS-20.

In another embodiment the surfactant is at a concentration of about 0.05 to 0.1 mg.

In another embodiment the surfactant is at a concentration of about 0.5 to 1 mg.

In another embodiment the concentration ratio of anti-PD-1 antibody to disaccharide is 1:1.

In another embodiment the lyosphere is reconstituted (a reconstituted lyosphere formulation).

### (c) Reconstituted Lyosphere Formulations

The invention further provides a reconstituted lyosphere formulation (to be administered to a patient) comprising: a) 130 mg/mL to 200 mg/mL of an anti-PD-1 antibody: b) 130 mg/mL to 200 mg/mL stabilizer (disaccharide); c) 1 to 20 mM buffer, pH 5.0-6.5; and d) about 0.02-0.04% surfactant.

In one embodiment the pH of the reconstituted lyosphere formulation is about 5.0 to about 6.5.

In another embodiment the pH of the reconstituted lyosphere formulation is about 5.5 to about 6.0.

In another embodiment the reconstituted lyosphere formulation further comprises a viscosity reducer.

In another embodiment the viscosity reducer is arginine, lactic acid, or nicotinic acid and/or combinations thereof.

In one embodiment the anti-PD-1 antibody is pembrolizumab.

In one embodiment the anti-PD-1 antibody is nivolumab.

In a further embodiment the anti-PD-1 antibody is atezolizumab.

In yet another embodiment, the anti-PD-1 antibody is cemiplimab.

In still another embodiment the anti-PD-1 antibody is avelumab.

In a specific embodiment the anti-PD-1 antibody is dostarlimab.

In another embodiment the anti-PD-1 antibody is pidilizumab.

In one embodiment the stabilizer (disaccharide) is sucrose or trehalose, or a combination of sucrose and trehalose.

In one embodiment the stabilizer (disaccharide) is a combination of sucrose and trehalose, wherein the concentration of trehalose is greater than concentration of sucrose.

In one embodiment the buffer is selected from the group consisting of potassium phosphate, sodium phosphate, sodium acetate, histidine, imidazole, sodium citrate, sodium succinate, ammonium bicarbonate, HEPES, Tris, Bis-Tris, and a carbonate, and/or combinations thereof.

In another embodiment the buffer is histidine.

In another embodiment the surfactant is a non-ionic surfactant.

In another embodiment the surfactant is polysorbate 80 (PS-80), or polysorbate 20 (PS-20).

In one embodiment the surfactant is PS-80.

In one embodiment the surfactant is PS-20.

In another embodiment the reconstituted lyosphere formulation (to be administered to a patient) comprises: a) 130 mg/mL to 200 mg/mL of an anti-PD-1 antibody: b) 130 mg/mL to 200 mg/mL trehalose, or about 130 mg/mL to 200 mg/mL sucrose, or a combination of trehalose and sucrose totaling about 130 mg/mL to 200 mg/mL; c) 10 mM histidine buffer, pH 5.5-6.0; and d) about 0.02-0.04% w/v PS80.

### Anti-PD-1 Antibodies

The invention provides stable formulations (a lyosphere or a reconstituted lyosphere formulation) comprising antibodies or antigen binding fragments thereof, which specifically bind to human PD-1 (e.g, a human or humanized anti-PD-1 antibody) as the active pharmaceutical ingredient (API), as well as methods for using the pharmaceutical formulations of the invention and methods of making the pharmaceutical formulations of the invention. Any anti-PD-1 antibody or antigen binding fragment thereof can be used in the formulations, pharmaceutical formulations and methods of the invention. In particular embodiments, the API is an anti-PD-1 antibody, which is selected from pembrolizumab, nivolumab, atezolizumab, cemiplimab, avelumab, dostarlimab, and pidilizumab, or any variant or antigen biding fragment of any such antibody thereof. In specific embodiments, the anti-PD-1 antibody is pembrolizumab or a pembrolizumab variant, or a binding fragment of pembrolizumab or a pembrolizumab variant. In specific embodiments, the anti-PD-1 antibody is pembrolizumab. In alternative embodiments, the anti-PD-1 antibody is nivolumab or a nivolumab variant. Table 1 provides amino acid sequences for exemplary anti-human PD-1 antibodies pembrolizumab and nivolumab. Alternative PD-1 antibodies and antigen-binding fragments that are useful in the formulations, pharmaceutical formulations and methods of the invention are shown in Table 2.

In some embodiments, an anti-human PD-1 antibody or antigen binding fragment thereof for use in the formulations and pharmaceutical formulations of the invention comprises three light chain CDRs of CDRL1, CDRL2 and CDRL3 and/or three heavy chain CDRs of CDRH1, CDRH2 and CDRH3.

In one embodiment of the invention, CDRL1 is SEQ ID NO:1 or a variant of SEQ ID NO:1, CDRL2 is SEQ ID NO:2 or a variant of SEQ ID NO:2, and CDRL3 is SEQ ID NO:3 or a variant of SEQ ID NO:3.

In one embodiment, CDRH1 is SEQ ID NO:6 or a variant of SEQ ID NO:6, CDRH2 is SEQ ID NO: 7 or a variant of SEQ ID NO: 7, and CDRH3 is SEQ ID NO:8 or a variant of SEQ ID NO:8.

In one embodiment, the three light chain CDRs 1, 2 and 3 are SEQ ID NO: 1, SEQ ID NO:2, and SEQ ID NO:3, respectively, and the three heavy chain CDRs 1, 2 and 3 are SEQ ID NO:6, SEQ ID NO:7 and SEQ ID NO:8, respectively.

In an alternative embodiment of the invention, CDRL1 is SEQ ID NO:11 or a variant of SEQ ID NO:11, CDRL2 is SEQ ID NO:12 or a variant of SEQ ID NO:12, and CDRL3 is SEQ ID NO:13 or a variant of SEQ ID NO: 13.

In one embodiment, CDRH1 is SEQ ID NO: 16 or a variant of SEQ ID NO: 16.

CDRH2 is SEQ ID NO:17 or a variant of SEQ ID NO: 17, and CDRH3 is SEQ ID NO:18 or a variant of SEQ ID NO: 18.

In one embodiment, the three light chain CDRs 1, 2 and 3 are SEQ ID NO: 1, SEQ ID NO:2, and SEQ ID NO:3, respectively, and the three heavy chain CDRs 1, 2 and 3 are SEQ ID NO:6, SEQ ID NO:7 and SEQ ID NO:8, respectively.

In an alternative embodiment, the three light chain CDRs 1, 2 and 3 are SEQ ID NO: 11. SEQ ID NO:12, and SEQ ID NO: 13, respectively, and the three heavy chain CDRs 1, 2 and 3 are SEQ ID NO: 16, SEQ ID NO: 17 and SEQ ID NO: 18, respectively.

In a further embodiment of the invention, CDRL1 is SEQ ID NO:21 or a variant of SEQ ID NO:21, CDRL2 is SEQ ID NO:22 or a variant of SEQ ID NO:22, and CDRL3 is SEQ ID NO:23 or a variant of SEQ ID NO:23.

In yet another embodiment, CDRH1 is SEQ ID NO:24 or a variant of SEQ ID NO:24, CDRH2 is SEQ ID NO: 25 or a variant of SEQ ID NO:25, and CDRH3 is SEQ ID NO:26 or a variant of SEQ ID NO: 26.

In another embodiment, the three light chain CDRs 1, 2 and 3 are SEQ ID NO:21, SEQ ID NO:22, and SEQ ID NO:23, respectively, and the three heavy chain CDRs 1, 2 and 3 are SEQ ID NO:24, SEQ ID NO:25 and SEQ ID NO:26, respectively.

Some antibody and antigen binding fragments of the formulations (the pre-lyosphere formulations) and pharmaceutical formulations (the lyospheres and the reconstituted lyosphere formulations) of the invention comprise a light chain variable region and a heavy chain variable region. In some embodiments, the light chain variable region comprises SEQ ID NO:4 or a variant of SEQ ID NO:4, and the heavy chain variable region comprises SEQ ID NO:9 or a variant of SEQ ID NO:9. In further embodiments, the light chain variable region comprises SEQ ID NO: 14 or a variant of SEQ ID NO:14, and the heavy chain variable region comprises SEQ ID NO: 19 or a variant of SEQ ID NO: 19. In further embodiments, the heavy chain variable region comprises SEQ ID NO:27 or a variant of SEQ ID NO:27 and the light chain variable region comprises SEQ ID NO:28 or a variant of SEQ ID NO:28, SEQ ID NO:29 or a variant of SEQ ID NO:29, or SEQ ID NO:30 or a variant of SEQ ID NO:30. In such embodiments, a variant light chain or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the formulations and pharmaceutical formulations of the invention, the antibody or antigen binding fragment comprises a light chain variable region comprising or consisting of SEQ ID NO:4 and a heavy chain variable region comprising or consisting SEQ ID NO:9. In a further embodiment, the antibody or antigen binding fragment comprises a light chain variable region comprising or consisting of SEQ ID NO:14 and a heavy chain variable region comprising or consisting of SEQ ID NO:19. In one embodiment of the formulations and pharmaceutical formulations of the invention, the antibody or antigen binding fragment comprises a light chain variable region comprising or consisting of SEQ ID NO:28 and a heavy chain variable region comprising or consisting SEQ ID NO:27. In a further embodiment, the antibody or antigen binding fragment comprises a light chain variable region comprising or consisting of SEQ ID NO:29 and a heavy chain variable region comprising or consisting SEQ ID NO:27. In another embodiment, the antibody or antigen binding fragment comprises a light chain variable region comprising or consisting of SEQ ID NO:30 and a heavy chain variable region comprising or consisting SEQ ID NO:27.

In another embodiment, the formulations of the invention comprise an antibody or antigen binding protein that has a V_{L} domain and/or a V_{H} domain with at least 95%, 90%, 85%, 80%, 75% or 50% sequence homology to one of the V_{L} domains or V_{H} domains described above, and exhibits specific binding to PD-1. In another embodiment, the antibody or antigen binding protein of the formulations and pharmaceutical formulations of the invention comprises V_{L} and V_{H} domains having up to 1, 2, 3, 4, or 5 or more amino acid substitutions, and exhibits specific binding to PD-1.

In any of the embodiments above, the API may be a full-length anti-PD-1 antibody or an antigen binding fragment thereof that specifically binds human PD-1. In certain embodiments, the API is a full-length anti-PD-1 antibody selected from any class of immunoglobulins, including IgM.

IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In embodiments of the invention, the API is an anti-PD-1 antibody comprising a light chain comprising or consisting of a sequence of amino acid residues as set forth in SEQ ID NO:5 and a heavy chain comprising or consisting of a sequence of amino acid residues as set forth in SEQ ID NO:10. In alternative embodiments, the API is an anti-PD-1 antibody comprising a light chain comprising or consisting of a sequence of amino acid residues as set forth in SEQ ID NO: 15 and a heavy chain comprising or consisting of a sequence of amino acid residues as set forth in SEQ ID NO:20. In further embodiments, the API is an anti-PD-1 antibody comprising a light chain comprising or consisting of a sequence of amino acid residues as set forth in SEQ ID NO:32 and a heavy chain comprising or consisting of a sequence of amino acid residues as set forth in SEQ ID NO:31. In additional embodiments, the API is an anti-PD-1 antibody comprising a light chain comprising or consisting of a sequence of amino acid residues as set forth in SEQ ID NO:33 and a heavy chain comprising or consisting of a sequence of amino acid residues as set forth in SEQ ID 30 NO:31. In yet additional embodiments, the API is an anti-PD-1 antibody comprising a light chain comprising or consisting of a sequence of amino acid residues as set forth in SEQ ID NO:34 and a heavy chain comprising or consisting of a sequence of amino acid residues as set forth in SEQ ID NO:31. In some formulations of the invention, the API is pembrolizumab or a pembrolizumab biosimilar. In some formulations of the invention, the API is nivolumab or a nivolumab biosimilar.

Ordinarily, amino acid sequence variants of the anti-PD-1 antibodies and antigen binding fragments of the invention will have an amino acid sequence having at least 75% amino acid sequence identity with the amino acid sequence of a reference antibody or antigen binding fragment (e.g. heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410: Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70: ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358. Natl. Biomed. Res. Found., Washington, DC; Altschul, S. F., (1991) J. Mol. Biol. 219:555-565; States, D. J., et al. (1991) Methods 3:66-70: Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268: Karlin. S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the formulations, pharmaceutical formulations and methods herein. Specifically, kappa, lambda, or variants thereof are useful in the present formulations, pharmaceutical formulations and methods.

TABLE 1
Exemplary Anti-PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWY | 4 |
| Region | QQKPGQAPRLLIYLASYLESGVPARFSGSGSGTDFTLTISS | |
| | LEPEDFAVYYCQHSRDLPLTFGGGTKVEIK | |
| Light Chain | EIVLTQSPATLSLSPGERATLSCRASKGVSTSGYSYLHWY | 5 |
| | QQKPGQAPRLLIYLASYLESGVPARFSGSGSGTDFTLTISS | |
| | LEPEDFAVYYCQHSRDLPLTFGGGTKVEIKRTVAAPSVFI | |
| | FPPSDEQLKSGTASVVCLLNNFYPREAKVQWKVDNALQ | |
| | SGNSQESVTEQDSKDSTYSLSSTLTLSKADYEKHKVYAC | |
| | EVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWV | 9 |
| Region | RQAPGQGLEWMGGINPSNGGTNFNEKFKNRVTLTTDSST | |
| | TTAYMELKSLQFDDTAVYYCARRDYRFDMGFDYWGQG | |
| | TTVTVSS | |
| Heavy | QVQLVQSGVEVKKPGASVKVSCKASGYTFTNYYMYWV | 10 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGK | |

Nivolumab Light Chain

| | | |
|---|---|---|
| CDR1 | RASQSVSSYLA | 11 |
| CDR2 | DASNRAT | 12 |
| CDR3 | QQSSNWPRT | 13 |
| Variable | EIVLTQSPATLSLSPGERATLSCRASQSVSSYLAWYQQKP | 14 |
| Region | GQAPRLLIYDASNRATGIPARFSGSGSGTDFTLTISSLEPE | |
| | DFAVYYCQQSSNWPRTFGQGTKVEIK | |
| Light Chain | EIVLTQSPATLSLSPGERATLSCRASQSVSSYLAWYQQKP | 15 |
| | GQAPRLLIYDASNRATGIPARFSGSGSGTDFTLTISSLEPE | |
| | DFAVYYCQQSSNWPRTFGQGTKVEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |

Nivolumab Heavy Chain

| | | |
|---|---|---|
| CDR1 | NSGMH | 16 |
| CDR2 | VIWYDGSKRYYADSVKG | 17 |
| CDR3 | NDDY | 18 |
| Variable | QVQLVESGGGVVQPGRSLRLDCKASGITFSNSGMHWVR | 19 |
| Region | QAPGKGLEWVAVIWYDGSKRYYADSVKGRFTISRDNSK | |
| | NTLFLQMNSLRAEDTAVYYCATNDDYWGQGTLVTVSS | |
| Heavy | QVQLVESGGGVVQPGRSLRLDCKASGITFSNSGMHWVR | 20 |
| Chain | QAPGKGLEWVAVIWYDGSKRYYADSVKGRFTISRDNSK | |
| | NTLFLQMNSLRAEDTAVYYCATNDDYWGQGTLVTVSSA | |
| | STKGPSVFPLAPCSRSTSESTAALGCLVKDYFPEPVTVSW | |
| | NSGALTSGVHTFPAVLQSSGLYSLSSVVTVPSSSLGTKTY | |
| | TCNVDHKPSNTKVDKRVESKYGPPCPPCPAPEFLGGPSVF | |
| | LFPPKPKDTLMISRTPEVTCVVVDVSQEDPEVQFNWYVD | |
| | GVEVHNAKTKPREEQFNSTYRVVSVLTVLHQDWLNGKE | |
| | YKCKVSNKGLPSSIEKTISKAKGQPREPQVYTLPPSQEEM | |
| | TKNQVSLTCLVKGFYPSDIAVEWESNGQPENNYKTTPPV | |
| | LDSDGSFFLYSRLTVDKSRWQEGNVFSCSVMHEALHNH | |
| | YTQKSLSLSLGK | |

TABLE 2
Additional Anti-PD-1 Antibodies and Antigen Binding Fragments
Useful in the Formulations, Methods and Uses of the Invention.
A. Antibodies and antigen binding fragments comprising light and heavy
chain CDRs of hPD-1.08A in WO2008/156712

| | |
|---|---|
| CDRL1 | SEQ ID NO: 21 |
| CDRL2 | SEQ ID NO: 22 |
| CDRL3 | SEQ ID NO: 23 |
| CDRH1 | SEQ ID NO: 24 |
| CDRH2 | SEQ ID NO: 25 |
| CDRH3 | SEQ ID NO: 26 |

B. Antibodies and antigen binding fragments comprising the mature
h109A heavy chain variable region and one of the mature K09A light
chain variable regions in WO 2008/156712

| | |
|---|---|
| Heavy chain VR | SEQ ID NO: 27 |
| Light chain VR | SEQ ID NO: 28 or |
| | SEQ ID NO: 29 or |
| | SEQ ID NO: 30 |

C. Antibodies and antigen binding fragments comprising the mature 409
heavy chain and one of the mature K09A light chains in WO 2008/156712

| | |
|---|---|
| Heavy chain | SEQ ID NO: 31 |
| Light chain | SEQ ID NO: 32 or |
| | SEQ ID NO: 33 or |
| | SEQ ID NO: 34 |

In some embodiments of the formulations and pharmaceutical formulations of the invention, the API (e.g. pembrolizumab) is present in a concentration of about 25 mg/mL to about 200 mg/mL. In alternative embodiments, the API is present in a concentration of about 25 mg/mL, about 50 mg/mL, about 75 mg/mL, about 100 mg/mL, about 125 mg/mL, about 130 mg/mL about 150 mg/mL, about 165 mg/mL, about 167 mg/mL about 175 mg/mL, about 200 mg/mL.

In one embodiment, the API is present in a concentration of about 165 to about 170 mg/mL.

In one embodiment, the API is present in a concentration of about 167 mg/mL.

In one embodiment, the API is present in a concentration of about 130 mg/mL.

In additional embodiments, the API is present in a concentration of from about 25 mg/mL to about 75 mg/mL, from about 50 mg/mL to about 200 mg/mL; from about 75 mg/mL to about 200 mg/mL; from about 100 mg/mL to about 200 mg/mL; from about 25 mg/mL to about 175 mg/mL; from about 50 mg/mL to about 175 mg/mL; from about 75 mg/mL to about 175 mg/mL: from about 100 mg/mL to about 175 mg/mL; from about 25 mg/mL to about 150 mg/mL; from about 50 mg/mL to about 150 mg/mL; from about 75 mg/mL to about 150 mg/mL; from about 100 mg/mL to about 150 mg/mL; from about 25 mg/mL to about 125 mg/mL; from about 50 mg/mL to about 125 mg/mL: from about 75 mg/mL to about 125 mg/mL: from about 25 mg/mL to about 100 mg/mL, from about 125 mg/mL to about 175 mg/mL, from about 125 mg/mL to about 200 mg/mL, or from about 5 mg/mL to 200 mg/mL.

### Formulation and Pharmaceutical Formulation Excipients

The formulations and pharmaceutical formulations of the invention comprise at least one excipient that stabilizes the formulation and pharmaceutical formulation. In some embodiments, the formulation and pharmaceutical formulation comprises more than one stabilizer.

In some embodiments of the formulations and pharmaceutical formulations of the invention, the stabilizer is a disaccharide. In further embodiments, the disaccharide is sucrose. In additional embodiments, the disaccharide is trehalose. In still further embodiments, the disaccharide is a combination of sucrose and trehalose.

In some embodiments, the anti-human PD-1 antibody formulations and pharmaceutical formulations of the invention comprise a stabilizer selected from the group consisting of about 20% to about 30% weight/volume (w/v) sucrose, trehalose or a combination of sucrose and trehalose.

In some embodiments, the stabilizer is about 20% or 21%, or 22%, or 23%, or 24%, or 25%, or 26%, or 27%, or 28%, or 29%, or 30% weight/volume (w/v) sucrose or trehalose or a combination of sucrose and trehalose.

In some embodiments the amount of trehalose is greater than the amount of sucrose.

In certain embodiments, the formulations and pharmaceutical formulations of the invention comprise arginine, e.g., L-arginine or a pharmaceutically acceptable salt thereof. In additional embodiments, the formulations and pharmaceutical formulations of the invention comprise arginine hydrochloride (i.e., arginine HCl). In further embodiments, the formulations and pharmaceutical formulations comprise arginine succinate. In further embodiments, the arginine is L-arginine.

The formulations and pharmaceutical formulations of the invention optionally comprise arginine, e.g., L-arginine, or a pharmaceutically acceptable salt thereof, which may provide additional stability to the formulation and pharmaceutical formulation, as well as control viscosity, which allows a formulation and a pharmaceutical formulation at high API concentration. In some embodiments of the invention, the L-arginine or pharmaceutically acceptable salt is present in the formulations and pharmaceutical formulations in an amount of 0.25% to about 3% weight/volume. In additional embodiments, the L-arginine or pharmaceutically acceptable salt is present in an amount of about 0.25% w/v, about 0.50% w/v, about 0.75% w/v, about 1.0% w/v, about 1.25% w/v, about 1.5% w/v, about 1.75% w/v, about 2.0% w/v, about 2.25% w/v, about 2.5% w/v, about 2.75% w/v or about 3.0% w/v. In further embodiments, the L-arginine or pharmaceutically acceptable salt is present in an amount of about 0 to about 2.75% w/v, 0 to about 2.5% w/v, 0 to about 2.25% w/v, 0 to about 2% w/v, 0 to about 1.75% w/v, 0 to about 1.5% w/v, 0 to about 1.25% w/v, 0 to about 1.0% w/v, about 0.5% to about 3.0% w/v, about 0.5% to about 2.75% w/v, about 0.5% to about 2.5% w/v, about 0.5% to about 2.25% w/v, about 0.5% to about 2% w/v, about 0.5% to about 1.75% w/v, about 0.5% to about 1.5% w/v, about 0.5% to about 1.25% w/v, about 0.5% to about 1.0% w/v, about 1.0% to about 3.0% w/v, about 1.0% to about 2.75% w/v, about 1.0% to about 2.5% w/v, about 1.0% to about 2.25% w/v, about 1.0% to about 2% w/v, about 1.0% to about 1.75%-w/v, about 1.0% to about 1.5% w/v, about 1.5% to about 3.0% w/v, about 1.5% to about 2.75% w/v, about 1.5% to about 2.5% w/v, about 1.5% to about 2.25% w/v, about 1.5% to about 2% w/v, or about 2% to about 3% w/v.

As noted above, in specific embodiments, the formulations and pharmaceutical formulations of the invention may also comprise arginine, e.g., L-arginine, or a pharmaceutically acceptable salt thereof, e.g., an amount of L-arginine from about 0.25% to about 3.0% w/v.

In addition to an anti-PD-1 antibody (for example pembrolizumab), and a stabilizer (disaccharide) in the amounts/concentrations specified above, the formulations and pharmaceutical formulations of the invention also comprise a buffer. In some embodiments the buffer is present in an amount of about 5 mM to about 20 mM, which provides for a pH in the range of about 4.5 to 6.4.

In some embodiments of the invention, the buffer provides the formulation and pharmaceutical formulation with a pH in the range from about 4.5 to about 6.5. In further embodiments, the pH is in a range of about 5.0 to about 6.0. In still further embodiments, the pH is from about 5.3 to about 5.8. In other embodiments, the pH is from about 6.0 to about 6.4.

In particular embodiments, the buffer provides a pH of the formulation of about 5.0, about 5.1, about 5.2, about 5.3, about 5.4, about 5.5, about 5.6, about 5.7, about 5.8, about 5.9, about 6.0, about 6.2 or about 6.4. Examples of buffers that will control the pH in this range include succinate (sodium or potassium), histidine, sodium acetate, phosphate (sodium or potassium), Tris (tris (hydroxymethyl) aminomethane), diethanolamine, citrate (sodium) and other organic acid buffers.

In specific embodiments of the invention, the buffer is histidine at a pH of about 5.0 to about 6.0. In some embodiments, the buffer is an L-histidine buffer.

When a range of pH values is recited, such as “a pH between pH 5.5 and 6.0,” the range is intended to be inclusive of the recited values. Unless otherwise indicated, the pH refers to the pH after reconstitution of the lyosphere of the invention (i.e., the reconstituted lyosphere formulation). The pH is typically measured at 25° C. using standard glass bulb pH meter. As used herein, a solution comprising “histidine buffer at pH X” refers to a solution at pH X and comprising the histidine buffer, i.e., the pH is intended to refer to the pH of the solution.

In addition to an anti-PD-1 antibody (for example, pembrolizumab), a stabilizer (disaccharide), and a buffer in the amounts/concentrations specified above, the formulations and pharmaceutical formulations of the invention may also comprise an anti-oxidant. In embodiments of the invention, the anti-oxidant is methionine. In embodiments of the invention, the anti-oxidant is L-methionine, or a pharmaceutically acceptable salt thereof. In further embodiments, the methionine is L-methionine. In other embodiments, the anti-oxidant is L-methionine HCl. In other embodiments, the anti-oxidant is histidine.

In some embodiments, the anti-oxidant (e.g. L-methionine) is present in the formulations and pharmaceutical formulations of the invention in an amount of about 1 mM to about 20 mM. In further embodiments, the anti-oxidant is present in an amount of about 5 mM to about 20 mM, about 5 mM to about 15 mM, about 5 mM to about 10 mM. In additional embodiments, the anti-oxidant is present in an amount of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM.

In embodiments wherein the anti-oxidant is histidine, the histidine can be present in amounts up to 100 mM. In such embodiments, histidine can serve as a buffer and as an anti-oxidant in the formulations and pharmaceutical formulations of the invention.

In addition to an anti-PD-1 antibody (for example, pembrolizumab), a stabilizer (disaccharide), a buffer, and optionally an anti-oxidant in the amounts/concentrations specified above, the formulations and pharmaceutical formulations of the invention also comprise a surfactant. Surfactants are typically added to formulations and pharmaceutical formulations to provide stability, reduce and/or prevent aggregation or to prevent and/or inhibit protein damage during processing conditions such as purification, filtration, freeze-drying, transportation, storage, and delivery. In some embodiments of the invention, a surfactant is useful for providing additional stability to the active ingredient(s), i.e., the anti-PD-1 antibody (for example pembrolizumab).

Surfactants that may be useful in the formulations and pharmaceutical formulations of the invention include, but are not limited to: nonionic surfactants such as polyoxyethylene sorbitan fatty acid esters (Polysorbates, sold under the trade name Tween@) (Uniquema Americas LLC, Wilmington, DE)) including Polysorbate-20 (polyoxyethylene sorbitan monolaurate), Polysorbate-40 (polyoxyethylene sorbitan monopalmitate), Polysorbate-60 (polyoxyethylene sorbitan monostearate), and Polysorbate-80 (polyoxyethylene sorbitan monooleate): polyoxyethylene alkyl ethers such as Brijg 58 (Uniquema Americas LLC, Wilmington, DE) and Brij(R 35; poloxamers (e.g., poloxamer 188); Triton®X-100 (Union Carbide Corp., Houston, TX) and Tritong X-114; NP40; Span 20, Span 40, Span 60, Span 65, Span 80 and Span 85; copolymers of ethylene and propylene glycol (e.g., the Pluronic® series of nonionic surfactants such as Pluronic®R) F68. Pluronic®1ORS. Pluronic®F108, pluronicV F127, Pluronic® F38, pluronicR L44, Pluronic®L62 (BASF Corp., Ludwigshafen, Germany); and sodium dodecyl sulfate (SDS).

The amount of surfactant to be included in the formulations and pharmaceutical formulations of the invention is an amount sufficient to perform the desired function, i.e., a minimal amount necessary to stabilize the active pharmaceutical ingredient (i.e., the anti-PD-1 antibody) in the formulation and pharmaceutical formulation. Typically, the surfactant is present in a concentration of from about 0.008% to about 0.1% w/v. In some embodiments of this aspect of the invention, the surfactant is present in the formulation and pharmaceutical formulation in an amount from about 0.01% to about 0.04%; from about 0.01% to about 0.03%, from about 0.01% to about 0.02%, from about 0.015% to about 0.04%; from about 0.015% to about 0.03%, from about 0.015% to about 0.02%, from about 0.02% to about 0.04%, from about 0.02% to about 0.035%, or from about 0.02% to about 0.03%. In specific embodiments, the surfactant is present in an amount of about 0.02%. In alternative embodiments, the surfactant is present in an amount of about 0.01%, about 0.015%, about 0.025%, about 0.03%, about 0.035%, or about 0.04%.

In exemplary embodiments of the invention, the surfactant is a nonionic surfactant selected from the group consisting of polysorbate 20 (PS-20), polysorbate 80 (PS-80) and F127. In preferred embodiments, the surfactant is PS-80.

In specific embodiments, the anti-PD-1 formulations and pharmaceutical formulations of the invention comprise about 0.01% to about 0.04% PS-80. In further embodiments, the formulations and pharmaceutical formulations of the invention comprise PS-80 in an amount of about 0.008%, about 0.01%, about 0.015%, about 0.02%, about 0.025%, about 0.03%, about 0.035%, about 0.04% or about 0.045%. In particular embodiments, the formulations and pharmaceutical formulations of the invention comprise about 0.02% PS-80.

### SPECIFIC ASPECTS AND EMBODIMENTS OF THE INVENTION

The present disclosure provides pre-lyosphere formulations, lyospheres and reconstituted lyosphere formulations with high anti-PD-1 antibody and disaccharide content and methods of making and using the same.

In an embodiment, the invention provides a pre-lyosphere formulation comprising: a) at least about 25 mg/mL of an anti-PD-1 antibody; b) at least about 25 mg/mL stabilizer (disaccharide); c) a buffer, and d) a surfactant.

In one embodiment the anti-PD-1 antibody is pembrolizumab or a pembrolizumab variant.

In another embodiment the anti-PD-1 antibody is nivolumab or a nivolumab variant.

In some embodiments, the PD-1 antagonist is selected from the group consisting of: pembrolizumab, nivolumab, atezolizumab, durvalumab, cemiplimab, avelumab, dostarlimab, and pidilizumab.

In certain embodiments, the anti-PD-1 antibody is pembrolizumab.

In other embodiments the anti-PD-1 antibody is nivolumab.

In further embodiments the anti-PD-1 antibody is atezolizumab.

In yet other embodiments, the anti-PD-1 antibody is cemiplimab.

In still other embodiments the anti-PD-1 antibody is avelumab.

In specific embodiments the anti-PD-1 antibody is dostarlimab.

In specific embodiments the anti-PD-1 antibody is pidilizumab.

In another embodiment the stabilizer (disaccharide) is sucrose or trehalose, or a combination of sucrose and trehalose.

In another embodiment, the stabilizer (disaccharide) is a combination of sucrose and trehalose, wherein the concentration of trehalose is greater than concentration of sucrose.

In another embodiment the anti-PD-1 antibody is at a concentration of 25 mg/mL to 200 mg/mL, or at a concentration of 50 mg/mL to 200 mg/mL, or at a concentration of 100 mg/mL to 200 mg/mL, or at a concentration of 150 mg/mL to 200 mg/mL. In some embodiments the anti-PD-1 antibody is at a concentration of about 225 mg/mL, or about 250 mg/mL, or about 275 mg/mL, or about 300 mg/mL or greater.

In another embodiment the disaccharide is at a concentration of 25 mg/mL to 250 mg/mL, or at a concentration of 50 mg/mL to 200 mg/mL, or at a concentration of 100 mg/mL to 200 mg/mL, or at a concentration of 150 mg/mL to 200 mg/mL. In some embodiments the disaccharide is at a concentration of 200 mg/mL to 300 mg/mL. In some embodiments the disaccharide is at a concentration of about 210 mg/mL, or about 220 mg/mL, or about 230 mg/mL, or about 240 mg/mL, or about 250 mg/mL, or about 260 mg/mL, or about 270 mg/mL, or about 280 mg/mL, or about 290 mg/mL, or about 300 mg/mL.

In another embodiment the disaccharide is about 20% weight/volume (w/v), or about 25% w/v, or about 30% w/v.

In another embodiment the buffer is selected from the group consisting of potassium phosphate, sodium phosphate, sodium acetate, histidine, imidazole, sodium citrate, sodium succinate, ammonium bicarbonate, HEPES, Tris, Bis-Tris, and a carbonate, and/or combinations thereof.

In another embodiment the buffer is at a concentration of about 5-50 mM.

In another embodiment the buffer is at a concentration of about 5-15 mM.

In another embodiment the buffer is at a concentration of about 10-20 mM.

In another embodiment the buffer is at a concentration of about 10 mM.

In another embodiment the buffer is at a concentration of 10 mM.

In another embodiment the buffer is histidine.

In another embodiment the surfactant is a non-ionic surfactant.

In another embodiment the surfactant is a polysorbate 80 (PS-80), or a polysorbate 20 (PS-20).

In another embodiment the surfactant is PS-80.

In another embodiment the surfactant is PS-20.

In another embodiment the surfactant is at a concentration of about 0.01-0.2%.

In another embodiment the surfactant is at a concentration of about 0.02-0.04%.

In another embodiment the surfactant is at a concentration of about 0.1-2 mg/mL.

In another embodiment the surfactant is at a concentration of about 0.2-0.4 mg/mL.

In another embodiment the concentration ratio of anti-PD-1 antibody to disaccharide is 1:1.

In another embodiment the pH of the pre-lyosphere formulation is about 5.0 to about 6.5.

In another embodiment the pH of the pre-lyosphere formulation is about 5.5 to about 6.0.

In another embodiment the pre-lyosphere formulation further comprises a viscosity reducer.

In another embodiment the viscosity reducer is arginine, lactic acid, or nicotinic acid and/or combinations thereof.

In another embodiment the pre-lyosphere formulation comprises: a) about 25 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 50 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 75 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 100 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 125 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 150 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 175 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 200 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 225 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 250 mg/mL of pembrolizumab; b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 275 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another embodiment the pre-lyosphere formulation comprises: a) about 300 mg/mL of pembrolizumab: b) about 250 mg/mL trehalose, or about 250 mg/mL sucrose, or a combination of trehalose and sucrose totaling about 250 mg/mL; c) about 10 mM histidine buffer, pH 5.5-6.0; and d) about 0.02-0.04% PS-80.

In another aspect, the invention provides a lyosphere (50 pl volume/bead) comprising: a) 1.25 mg to 10 mg of an anti-PD-1 antibody; b) 1.25 mg to 12.5 mg of a stabilizer (disaccharide); and c) 0.005 to 0.01 mg of surfactant.

In an embodiment the anti-PD-1 antibody is pembrolizumab or a variant thereof.

In an embodiment the anti-PD-1 antibody is nivolumab or a variant thereof.

In some embodiments, the PD-1 antagonist is selected from the group consisting of: pembrolizumab, nivolumab, atezolizumab, durvalumab, cemiplimab, avelumab, dostarlimab, and pidilizumab.

In certain embodiments, the anti-PD-1 antibody is pembrolizumab.

In other embodiments the anti-PD-1 antibody is nivolumab.

In further embodiments the anti-PD-1 antibody is atezolizumab.

In yet other embodiments, the anti-PD-1 antibody is cemiplimab.

In still other embodiments the anti-PD-1 antibody is avelumab.

In specific embodiments the anti-PD-1 antibody is dostarlimab.

In specific embodiments the anti-PD-1 antibody is pidilizumab.

In an embodiment the anti-PD-1 antibody is at a concentration of about 1.25 mg, or about 2 mg, or about 4 mg, or about 6 mg, or about 8 mg, or about 10 mg.

In an embodiment the stabilizer (disaccharide) is at a concentration of about 1.25 mg, or about 2 mg, or about 4 mg, or about 6 mg, or about 8 mg, or about 10 mg, or about 12 mg, or about 12.5 mg.

In another embodiment the stabilizer (disaccharide) is sucrose or trehalose, or a combination of sucrose and trehalose.

In another embodiment the stabilizer (disaccharide) is a combination of sucrose and trehalose, wherein the concentration of trehalose is greater than concentration of sucrose.

In another embodiment the surfactant is a non-ionic surfactant.

In another embodiment the surfactant is a polysorbate 80 (PS-80), or a polysorbate 20 (PS-20).

In another embodiment the surfactant is PS-80.

In another embodiment the surfactant is PS-20.

In another embodiment the surfactant is at a concentration of about 0.05 to 0.1 mg.

In another embodiment the surfactant is at a concentration of about 0.5 to 1 mg.

In another embodiment the concentration ratio of anti-PD-1 antibody to disaccharide is 1:1.

In another embodiment the lyosphere is reconstituted (a reconstituted lyosphere formulation).

In another embodiment the pH of the reconstituted lyosphere formulation is about 5.0 to about 6.5.

In another embodiment the pH of the reconstituted lyosphere formulation is about 5.5 to about 6.0.

In another embodiment the reconstituted lyosphere formulation further comprises a viscosity reducer.

In another embodiment the viscosity reducer is arginine, lactic acid, or nicotinic acid and/or combinations thereof.

In another aspect, the invention provides a reconstituted lyosphere formulation (to be administered to a patient) comprising: a) 130 mg/mL to 200 mg/mL of the anti-PD-1 antibody: b) 130 mg/mL to 200 mg/mL stabilizer (disaccharide); c) 1 to 20 mM buffer, pH 5.0-6.5; and d) about 0.02-0.04% surfactant.

In an embodiment the anti-PD-1 antibody is pembrolizumab or a variant thereof.

In an embodiment the anti-PD-1 antibody is nivolumab or a variant thereof.

In some embodiments, the PD-1 antagonist is selected from the group consisting of: pembrolizumab, nivolumab, atezolizumab, durvalumab, cemiplimab, avelumab, dostarlimab, and pidilizumab.

In certain embodiments, the anti-PD-1 antibody is pembrolizumab.

In other embodiments the anti-PD-1 antibody is nivolumab.

In further embodiments the anti-PD-1 antibody is atezolizumab.

In yet other embodiments, the anti-PD-1 antibody is cemiplimab.

In still other embodiments the anti-PD-1 antibody is avelumab.

In specific embodiments the anti-PD-1 antibody is dostarlimab.

In specific embodiments the anti-PD-1 antibody is pidilizumab.

In another embodiment the stabilizer (disaccharide) is sucrose or trehalose, or a combination of sucrose and trehalose.

In another embodiment the stabilizer (disaccharide) is a combination of sucrose and trehalose, wherein the concentration of trehalose is greater than concentration of sucrose.

In another embodiment the buffer is selected from the group consisting of potassium phosphate, sodium phosphate, sodium acetate, histidine, imidazole, sodium citrate, sodium succinate, ammonium bicarbonate, HEPES, Tris, Bis-Tris, and a carbonate, and/or combinations thereof.

In another embodiment the buffer is histidine.

In another embodiment the surfactant is a non-ionic surfactant.

In another embodiment the surfactant is a polysorbate 80 (PS-80), or a polysorbate 20 (PS-20).

In another embodiment the surfactant is PS-80.

In another embodiment the surfactant is PS-20.

In another embodiment the reconstituted lyosphere formulation further comprises a viscosity reducer.

In another embodiment the viscosity reducer is arginine, lactic acid, or nicotinic acid and/or combinations thereof.

In another embodiment the reconstituted lyosphere formulation (to be administered to a patient) comprises: a) 130 mg/mL to 200 mg/mL of the anti-PD-1 antibody; b) 130 mg/mL to 200 mg/mL trehalose, or about 130 mg/mL to 200 mg/mL sucrose, or a combination of trehalose and sucrose totaling about 130 mg/mL to 200 mg/mb: c) 10 mM histidine buffer, pH 5.5-6.0; and d) about 0.02-0.04% PS80.

Lyospheres Lyospheres of the anti-PD-1 antibodies provide several advantages. Lyospheres in general offer better chemical stability than solution formulations, and thus increased half-life. A lyosphere may also be reconstituted at different concentrations depending on clinical factors, such as route of administration or dosing. For example, a lyosphere may be reconstituted at a high concentration (i.e., in a small volume) when necessary for subcutaneous administration. One such lyosphere is disclosed at WO2014/093206, which is hereby incorporated by reference in its entirety.

Typically, the lyosphere is prepared in anticipation of reconstitution at high concentration of drug product (DP, in an exemplary embodiment humanized anti-PD-1 antibody pembrolizumab, or antigen binding fragment thereof), i.e., in anticipation of reconstitution in a low volume of water. Subsequent dilution with water or isotonic buffer can then readily be used to dilute the DP to a lower concentration. Typically, excipients are included in a lyosphere of the invention at levels that will result in a roughly isotonic formulation when reconstituted at high DP concentration, e.g., for subcutaneous administration. Reconstitution in a larger volume of water to give a lower DP concentration will necessarily reduce the tonicity of the reconstituted solution, but such reduction may be of little significance in non-subcutaneous, e.g., intravenous, administration. If isotonicity is desired at lower DP concentration, the lyosphere may be reconstituted in the standard low volume of water and then further diluted with isotonic diluent, such as 0.9% sodium chloride.

In an embodiment of the invention, humanized anti-PD-1 antibody (for example, pembrolizumab) is formulated as a lyosphere for reconstitution and utilization for subcutaneous administration. In various embodiments, the lyosphere of the invention enables reconstitution of the anti-PD-1 antibody to high concentrations, such as about 25, 30, 40, 50, 60, 75, 100, 125, 150, 175, 200, 225, 250 or more mg/mL.

Lyospheres are by definition essentially dry, and thus the concept of concentration is not useful in describing them. Describing a lyosphere in the terms of the weight of the components in a unit dose vial is more useful, but is problematic because it varies for different doses or vial sizes. In describing the lyospheres of the invention, it is useful to express the amount of a component as the ratio of the weight of the component compared to the weight of the drug substance (DS) in the same sample (e.g, a vial). This ratio may be expressed as a percentage. Such ratios reflect an intrinsic property of the lyosphere of the invention, independent of vial size, dosing, and reconstitution protocol.

In other embodiments, the lyosphere of anti-human PD-1 antibody, or antigen binding fragment, is defined in terms of the pre-lyophilization solution (the pre-lyosphere formulation) used to make the lyosphere. In one embodiment the pre-lyophilization solution (the pre-lyosphere solution) comprises antibody, or antigen-binding fragment thereof, at a concentration of about 25 mg/mL or about 50 mg/mL and so on. Such pre-lyophilization solutions (the pre-lyosphere formulations) may be at pH 4.4-5.2 (including about 4.4, 4.5, 4.6, 4.7, 4.8, 4.9, 5.0, 5.1, and 5.2), e.g., about pH 4.8, or about pH 5.5.

In yet other embodiments, the reconstituted lyosphere formulation of anti-human PD-1 antibody, or antigen binding fragment, is defined in terms of the reconstituted solution generated from the lyosphere.

Reconstituted solutions may comprise antibody, or antigen-binding fragment thereof, at concentrations of about 25, 30, 40, 50, 60, 75, 80, 90 or 100 mg/mL or higher concentrations such as 150 mg/mL, 167 mg/mL, 200 mg/mL, or up to about 250 mg/mL. Such reconstituted solutions may be at about pH 5.5, or range from about pH 5.0 to about 6.0 The lyospheres of the invention are formed by lyophilization (freeze-drying) of a pre-lyophilization solution (the pre-lyosphere formulation). Freeze-drying is accomplished by freezing the pre-lyosphere formulation and subsequently subliming water at a temperature suitable for primary drying. Under this condition, the product temperature is below the eutectic point or the collapse temperature of the pre-lyosphere formulation. Typically, the shelf temperature for the primary drying will range from about −30 to 25° C. (provided the product remains frozen during primary drying) at a suitable pressure, ranging typically from about 50 to 250 mTorr. The pre-lyosphere formulation, size and type of the container holding the sample (e.g., glass vial) and the volume of liquid will dictate the time required for drying, which can range from a few hours to several days (e.g. 40-60 hrs). A secondary drying stage may be carried out at about 0-40° C., depending primarily on the type and size of container and the type of antibody employed. The secondary drying time is dictated by the desired residual moisture level in the product and typically takes at least about 5 hours. Typically, the moisture content of a lyosphere is less than about 5%, and preferably less than about 3%. The pressure may be the same as that employed during the primary drying step. Freeze-drying conditions can be varied depending on the pre-lyosphere formulation and vial size.

In some instances, it may be desirable to lyophilize the pre-lyosphere formulation in the container in which reconstitution of the lyosphere is to be carried out in order to avoid a transfer step. The container in this instance may, for example, be a 3, 5, 10, 20, 50 or 100 cc vial.

The lyospheres of the invention are reconstituted prior to administration to a patient. The antibody may be reconstituted at a concentration of about 25, 30, 40, 50, 60, 75, 80, 90 or 100 mg/mL or higher concentrations such as 150 mg/mL, 200 mg/mL, 250 mg/mL, or 300 mg/mL up to about 500 mg/mL. High antibody concentrations are particularly useful where subcutaneous delivery of the reconstituted lyosphere formulation is intended.

Reconstitution generally takes place at a temperature of about 25° C. to ensure complete hydration, although other temperatures may be employed as desired. The time required for reconstitution will depend, e.g., on the type of diluent, amount of excipient(s) and antibody.

Exemplary diluents include sterile water, bacteriostatic water for injection (BWFI), a pH buffered solution (e.g. phosphate-buffered saline), sterile saline solution, Ringer's solution or dextrose solution.

The invention provides a liquid anti-human PD-1 antibody reconstituted lyosphere formulation that is reconstituted from a lyosphere wherein the reconstituted solution comprises: a) about 125 mg/mL to about 175 mg/mL of an anti-human PD-1 antibody (for example, pembrolizumab): b) about 20% to 30% weight/volume (w/v) disaccharide wherein the disaccharide is sucrose or trehalose or a combination of both sucrose and trehalose; c) about 8 mM to about 12 mM histidine buffer; and d) about 0.01% to about 0.04% PS-80.

### Methods of Use

The invention also relates to a method of treating cancer in a subject, the method comprising administering an effective amount of the reconstituted lyosphere formulation of the invention to the subject. In a particular embodiment of this method, the reconstituted lyosphere formulation is administered to the subject by subcutaneous administration.

In any of the methods of the invention, the cancer can be selected from the group consisting of: melanoma, lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, salivary cancer, prostate cancer (e.g. hormone refractory prostate adenocarcinoma), pancreatic cancer, colon cancer, esophageal cancer, liver cancer, thyroid cancer, glioblastoma, glioma, and other neoplastic malignancies.

In one aspect, the invention relates to a method of treating cancer in a patient in need thereof, the method comprising administering to the subject an effective amount of a reconstituted lyosphere formulation of the invention, to the patient. In specific embodiments of this method, the composition is administered to the subject via intravenous administration. In specific embodiments of this method, the composition is administered to the subject via subcutaneous administration.

In some embodiments, reconstituted lyosphere formulation of the invention is administered to the patient once every three weeks for 12 weeks or more. In other embodiments, the composition of the invention or is administered to the patient once every three weeks for 15 weeks or more, 18 weeks or more, 21 weeks or more, 24 weeks or more, 27 weeks or more, 30 weeks or more, 33 weeks or more, 36 weeks or more, 39 weeks or more, 42 weeks or more, 45 weeks or more, 48 weeks or more. 51 weeks or more. 54 weeks or more. 57 weeks or more, 60 weeks or more, 63 weeks or more, 66 weeks or more, 69 weeks or more, 72 weeks or more, 75 weeks or more, 78 weeks or more, 81 weeks or more, 84 weeks or more, 87 weeks or more, or 90 weeks or more.

In other embodiments, a reconstituted lyosphere formulation of the invention is administered to the patient once every six weeks for 12 weeks or more. In other embodiments, the composition of the invention or is administered to the patient once every six weeks for 18 weeks or more, 24 weeks or more, 30 weeks or more, 36 weeks or more, 42 weeks or more, 48 weeks or more, 54 weeks or more. 60 weeks or more, 66 weeks or more, 72 weeks or more, 78 weeks or more, 84 weeks or more, 90 weeks or more, 96 weeks or more, 102 weeks or more, 108 weeks or more, 114 weeks or more, 120 weeks or more, 126 weeks or more, or 132 weeks or more.

In other embodiments of the invention, a reconstituted lyosphere formulation of the invention is administered to the patient intravenously or subcutaneously. In yet another embodiment, a reconstituted lyosphere formulation of the invention is delivered by inhalation or insufflation.

In some embodiments, the invention comprises a method of treating cancer in a human patient comprising administering a reconstituted lyosphere formulation of the invention to the patient.

In one embodiment, the invention comprises a method of treating melanoma in a human patient comprising administering a reconstituted lyosphere formulation of the invention to the patient. In a further embodiment, the melanoma is unresectable or metastatic. In vet a further embodiment, the melanoma is adjuvant melanoma. In specific embodiments, the melanoma is resected stage III melanoma.

In one embodiment, the invention comprises a method of treating metastatic non-small cell lung cancer (NSCLC) in a human patient comprising administering a reconstituted lyosphere formulation of the invention to the patient. In a further embodiment, the NSCLC is squamous. In alternative embodiments, the NSCLC is non-squamous. In yet a further embodiment, the method further comprises administering carboplatin-paclitaxel or nab-paclitaxel to the patient. In another embodiment, the patient has a tumor with high PD-L1 expression [(Tumor Proportion Score (TPS) 250%)] and was not previously treated with platinum-containing chemotherapy. In yet another embodiment, the patient has a tumor with PD-L1 expression (TPS 1%) and was previously treated with platinum-containing chemotherapy. In another embodiment, the patient had disease progression on or after receiving platinum-containing chemotherapy. In another embodiment, the patient has a tumor with PD-L1 expression (TPS 1%) and was not previously treated with platinum-containing chemotherapy. In yet a further embodiment, the PD-L1 TPS is determined by an FDA-approved test. In another embodiment, the patient's tumor has no EGFR or ALK genomic aberrations. In a different embodiment, the patient's tumor has an EGFR or ALK genomic aberration and had disease progression on or after receiving treatment for the EGFR or ALK aberration(s) prior to receiving the anti-PD-1 antibody, or antigen binding fragment thereof.

In one embodiment, the invention comprises a method of treating metastatic non-small cell lung cancer (NSCLC) in a human patient comprising: (1) administering a reconstituted lyosphere formulation of the invention to the patient, and (2) administering pemetrexed and carboplatin to the patient. In another embodiment, the patient was not previously treated with an anti-cancer therapeutic prior to starting the combination treatment regimen with the reconstituted lyosphere formulation, in combination with pemetrexed and carboplatin. In certain embodiments, the patient has nonsquamous non-small cell lung cancer. In yet another embodiment, pemetrexed is administered to the patient in an amount of 500 mg/m². In another embodiment, pemetrexed is administered to the patient via intravenous infusion every 21 days. In specific embodiments, the infusion time is about 10 minutes. In another embodiment, the invention further comprises administering about 400 μg to about 1000 μg of folic acid to the patient once per day, beginning about 7 days prior to administering pemetrexed to the patient and continuing until about 21 days after the patient is administered the last dose of pemetrexed. In certain embodiments the folic acid is administered orally. In another embodiment, the invention further comprises administering about I mg of vitamin B12 to the patient about 1 week prior to the first administration of pemetrexed and about every three cycles of pemetrexed administration (i.e., approximately every 9 weeks). In certain embodiments the vitamin B12 is administered intramuscularly. In another embodiment, the invention further comprises administering about 4 mg of dexamethasone to the patient twice a day on the day before, the day of, and the day after pemetrexed administration. In certain embodiments the dexamethasone is administered orally.

In one embodiment, the invention comprises a method of treating recurrent or metastatic head and neck squamous cell cancer (HNSCC) in a human patient comprising administering a composition comprising a reconstituted lyosphere formulation of the invention to the patient. In certain sub-embodiments, the patient has recurrent or metastatic HNSCC. In one embodiment, the patient was not previously treated with platinum-containing chemotherapy and the patient's tumor expresses PD-L1 (Combined Positive Score (CPS) >20).

In a sub-embodiment, the patient was previously treated with platinum-containing chemotherapy.

In certain embodiments, the patient had disease progression on or after platinum-containing chemotherapy.

In one embodiment, the invention comprises a method of treating refractory classical Hodgkin lymphoma (cHL) in a human patient comprising administering a reconstituted lyosphere formulation of the invention to the patient. In one embodiment, the invention comprises a method of treating classical Hodgkin lymphoma (cHL) in a human patient comprising administering a reconstituted lyosphere formulation of the invention to the patient, wherein the patient has relapsed after 3 or more lines of therapy for cHL.

In a sub-embodiment, the patient is an adult patient.

In alternative sub-embodiments, the patient is a pediatric patient.

In one embodiment, the invention comprises a method of treating locally advanced or metastatic urothelial carcinoma in a human patient comprising administering an effective amount of reconstituted lyosphere formulation of the invention to the patient.

In sub-embodiments, the patient is not eligible for cisplatin-containing chemotherapy.

In sub-embodiments, the patient has a tumor that expresses PD-L1. In some embodiments, the PD-L1 expression level is characterized by a CPS≥10.

In sub-embodiments, the patient has disease progression during or following platinum-containing chemotherapy or within 12 months of neoadjuvant or adjuvant treatment with platinum-containing chemotherapy.

In one embodiment, the invention comprises a method of treating unresectable or metastatic, microsatellite instability-high (MSI-H) or mismatch repair deficient solid tumors in a human patient comprising administering a reconstituted lyosphere formulation of the invention to the patient.

In a sub-embodiment, the patient had disease progression following prior anti-cancer treatment.

In one embodiment, the invention comprises a method of treating unresectable or metastatic, microsatellite instability-high (MSI-H) or mismatch repair deficient colorectal cancer in a human patient comprising administering a reconstituted lyosphere formulation of the invention to the patient.

In sub-embodiments, the patient had disease progression following prior treatment with a fluoropyrimidine, oxaliplatin, and irinotecan.

In one embodiment, the invention comprises a method of treating recurrent locally advanced or metastatic gastric cancer in a human patient comprising administering a reconstituted lyosphere formulation of the invention to the patient.

In one embodiment, the invention comprises a method of treating recurrent locally advanced or metastatic gastroesophageal junction adenocarcinoma in a human patient comprising administering a reconstituted lyosphere formulation of the invention to the patient.

In sub-embodiments, the patient's tumor expresses PD-L1 [Combined Positive Score (CPS)≥1].

In sub-embodiments, the patient has disease progression on or after two or more prior lines of therapy including fluoropyrimidine- and platinum-containing chemotherapy.

In sub-embodiments, the patient has disease progression on or after two or more prior lines of therapy including HER2/neu-targeted therapy.

In one embodiment, the invention comprises a method of treating cervical cancer in a human patient comprising administering a reconstituted lyosphere formulation of the invention to the patient. In a sub-embodiment, the patient has recurrent or metastatic cervical cancer.

In sub-embodiments, the patient had disease progression on or after chemotherapy.

In other sub-embodiments, the patient has a tumor that expresses PD-L1 [CPS≥1].

In one embodiment, the invention comprises a method of treating cancer in a human patient comprising administering a reconstituted lyosphere formulation of the invention to the patient, wherein the patient has a cancer selected from the group consisting of melanoma, non-small cell lung cancer, small cell lung cancer, head and neck cancer, urothelial cancer, breast cancer, gastrointestinal cancer, gastroesophageal junction adenocarcinoma, multiple myeloma, hepatocellular cancer, non-Hodgkin lymphoma, primary mediastinal large B-cell lymphoma, renal cancer, Hodgkin lymphoma, mesothelioma, ovarian cancer, esophageal cancer, anal cancer, biliary tract cancer, colorectal cancer, cervical cancer, endometrial cancer, cutaneous squamous cell cancer, thyroid cancer, prostate cancer, glioblastoma, Merkel cell carcinoma, and salivary cancer.

In one embodiment, the invention comprises a method of treating cancer in a human patient comprising administering a reconstituted lyosphere formulation of the invention to the patient, wherein the patient has a small-cell lung cancer. In a sub-embodiment, the patient has metastatic SCLC. In certain sub-embodiments, the patient was previously treated with platinum-based chemotherapy with disease progression on or after platinum-based chemotherapy and at least one other prior line of therapy. In certain sub-embodiments, the patient had disease progression on or after the platinum-based chemotherapy and at least one other prior line of therapy.

In one embodiment, the invention comprises a method of treating non-Hodgkin lymphoma in a human patient comprising administering a reconstituted lyosphere formulation of the invention to the patient. In a sub-embodiment, the non-Hodgkin lymphoma is mediastinal large B-cell lymphoma. In some embodiments, the non-Hodgkin lymphoma is primary mediastinal large B-cell lymphoma (PMBCL) that is refractory. In other embodiments, the patients have PMBCL and has relapsed after 2 or more prior lines of therapy.

In one embodiment, the invention comprises a method of treating breast cancer in a human patient comprising administering a reconstituted lyosphere formulation of the invention to the patient. In a sub-embodiment, the breast cancer is triple negative breast cancer. In a sub-embodiment, the breast cancer is ER+/HER2− breast cancer.

In one embodiment, the invention comprises a method of treating nasopharvngeal cancer in a human patient comprising administering a reconstituted lyosphere formulation of the invention to the patient.

In one embodiment, the invention comprises a method of treating thyroid cancer in a human patient comprising administering a reconstituted lyosphere formulation of the invention to the patient.

In one embodiment, the invention comprises a method of treating salivary cancer in a human patient comprising administering a reconstituted lyosphere formulation of the invention to the patient.

In one embodiment, the invention comprises a method of treating Merkel cell carcinoma (MCC) in a human patient comprising administering a reconstituted lyosphere formulation of the invention to the patient. In sub-embodiments the MCC is recurrent locally advanced or metastatic.

In one embodiment, the invention comprises a method of treating cancer in a human patient comprising administering a reconstituted lyosphere formulation of the invention to the patient, wherein the cancer is selected from the group consisting of: melanoma, non-small cell lung cancer, relapsed or refractory classical Hodgkin lymphoma, head and neck squamous cell carcinoma, cervical cancer, urothelial cancer, esophageal cancer, gastric cancer, primary mediastinal large B-cell lymphoma, and hepatocellular carcinoma.

In one embodiment, the invention comprises a method of treating cancer in a human patient comprising administering a reconstituted lyosphere formulation of the invention to the patient, wherein the cancer is a heme malignancy.

In one embodiment, the heme malignancy is selected from the group consisting of: acute lymphoblastic leukemia (ALL), acute myeloid leukemia (AML), chronic lymphocytic leukemia (CLL), chronic myeloid leukemia (CML), ddiffuse large B-cell lymphoma (DLBCL), EBV-positive DLBCL, primary mediastinal large B-cell lymphoma, T-cell/histiocyte-rich large B-cell lymphoma, follicular lymphoma, Hodgkin lymphoma (HL), mantle cell lymphoma (MCL), multiple myeloma (MM), myeloid cell leukemia-1 protein (MCL-1), myelodysplastic syndrome (MDS), non-Hodgkin lymphoma (NHL), and small lymphocytic lymphoma (SLL).

In one embodiment, the invention comprises a method of treating cancer in a human patient comprising administering a reconstituted lyosphere formulation of the invention to the patient, wherein the patient has a tumor with a high mutational burden.

In one embodiment, the invention comprises a method of treating hepatocellular carcinoma in a human patient comprising administering a reconstituted lyosphere formulation of the invention to the patient. In a sub-embodiment, the patient was previously treated with sorafenib.

In one embodiment, the invention comprises a method of treating renal cancer in a human patient comprising administering reconstituted lyosphere formulation of the invention to the patient. In sub-embodiments, the renal cancer is clear cell renal cell carcinoma.

In one embodiment, the invention comprises a method of treating esophageal cancer in a human patient comprising administering a reconstituted lyosphere formulation of the invention to the patient. In a sub-embodiment, the esophageal cancer is recurrent locally advanced or metastatic squamous cell carcinoma of the esophagus. In a further sub-embodiment, the patient had disease progression after one or more lines of systemic therapy. In a further sub-embodiment, the patient's tumors express PD-L1 [Combined Positive Score (CPS) >10].

In one embodiment, the invention comprises a method of treating ovarian carcinoma in a human patient comprising administering a reconstituted lyosphere formulation of the invention to the patient.

In one embodiment, the invention comprises a method of treating colorectal cancer in a human patient comprising administering a reconstituted lyosphere formulation of the invention to the patient.

In any of the methods of the invention described herein, the “reconstituted lyosphere formulation of the invention” can be any reconstituted lyosphere formulation described herein.

Malignancies that demonstrate improved disease-free and overall survival in relation to the presence of tumor-infiltrating lymphocytes in biopsy or surgical material, e.g., melanoma, colorectal, liver, kidney, stomach/esophageal, breast, pancreas, and ovarian cancer are encompassed in the methods and treatments described herein. Such cancer subtypes are known to be susceptible to immune control by T lymphocytes. Additionally, included are refractory or recurrent malignancies whose growth may be inhibited using the antibodies described herein.

In some embodiments, the compositions of the invention are administered to a subject having a cancer characterized by elevated expression of PD-L1 and/or PD-L2 in tested tissue samples, including ovarian, renal, colorectal, pancreatic, breast, liver, gastric, esophageal cancers, and melanoma. Additional cancers that can benefit from treatment with the compositions of the invention include those associated with persistent infection with viruses such as human immunodeficiency viruses, hepatitis viruses class A. B and C. Epstein Barr virus, human papilloma viruses that are known to be causally related to for instance Kaposi's sarcoma, liver cancer, nasopharyngeal cancer, lymphoma, cervical, vulval, anal, penile, and oral cancers.

Additional aspects include methods of using a reconstituted lyosphere formulation of the invention to treat a patient having an infection or infectious disease. Thus, the invention provides a method for treating chronic infection in a mammalian subject comprising administering a reconstituted lyosphere formulation of the invention to the subject. In some specific embodiments of this method, the composition is administered to the subject via intravenous administration. In other embodiments, the composition is administered to the subject by subcutaneous administration.

In this aspect, the compositions of the invention can be used alone, or in combination with vaccines, to stimulate the immune response to pathogens, toxins, and self-antigens. The compositions of the invention can be used to stimulate immune response to viruses infectious to humans, including but not limited to: human immunodeficiency viruses, hepatitis viruses class A, B and C, Epstein Barr virus, human cytomegalovirus, human papilloma viruses, and herpes viruses. Compositions of the invention that comprise antagonist anti-PD-1 antibodies or antibody fragments can be used to stimulate immune response to infection with bacterial or fungal parasites, and other pathogens. Viral infections with hepatitis B and C and HIV are among those considered to be chronic viral infections.

The reconstituted lyosphere formulations of the invention may be administered to a patient in combination with one or more “additional therapeutic agents”. The additional therapeutic agent may be a biotherapeutic agent (including but not limited to antibodies to VEGF, EGFR, Her2/neu, VEGF receptors, other growth factor receptors, CD20, CD40, CD-40L, OX-40, 4-1BB, and ICOS), a growth inhibitory agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

As noted above, in some embodiments of the methods of the invention, the method further comprises administering an additional therapeutic agent. In particular embodiments, the additional therapeutic agent is an anti-LAG3 antibody or antigen binding fragment thereof, an anti-GITR antibody, or antigen binding fragment thereof, an anti-TIGIT antibody, or antigen binding fragment thereof, an anti-CD27 antibody or antigen binding fragment thereof. In one embodiment, the additional therapeutic agent is a Newcastle disease viral vector expressing IL-12. In a further embodiment, the additional therapeutic agent is dinaciclib. In still further embodiments, the additional therapeutic agent is a STING agonist. In still further embodiments, the additional therapeutic agent is a PARP inhibitor. In still further embodiments, the additional therapeutic agent is a multi-tyrosine kinase inhibitor. In additional embodiments, the additional therapeutic agent is a MEK inhibitor. In additional embodiments, the additional therapeutic agent is a CXCR2 antagonist. In additional embodiments, the additional therapeutic agent is navarixin. In additional embodiments, the additional therapeutic agent is olarparib. In additional embodiments, the additional therapeutic agent is selumetinib. In additional embodiments, the additional therapeutic agent is axitinib.

Suitable routes of administration for the additional therapeutic agent may, for example, include parenteral delivery, including intramuscular, subcutaneous, as well as intrathecal, direct intraventricular, intravenous, intraperitoneal. Drugs can be administered in a variety of conventional ways, such as intraperitoneal, parenteral, intra-arterial or intravenous injection.

Selecting a dosage of the additional therapeutic agent depends on several factors, including the serum or tissue turnover rate of the entity, the level of symptoms, the immunogenicity of the entity, and the accessibility of the target cells, tissue or organ in the individual being treated. The dosage of the additional therapeutic agent should be an amount that provides an acceptable level of side effects. Accordingly, the dose amount and dosing frequency of each additional therapeutic agent (e.g. biotherapeutic or chemotherapeutic agent) will depend in part on the particular therapeutic agent, the severity of the cancer being treated, and patient characteristics. Guidance in selecting appropriate doses of antibodies, cytokines, and small molecules are available. See, e.g., Wawrzvnczak (1996) Antibody Therapy. Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J Med. 348:601-608; Milgrom et al. (1999)New Engl. J. Med. 341:1966-1973; Slamon et al. (2001) New Engl. J. Med. 344:783-792: Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Various literature references are available to facilitate selection of pharmaceutically acceptable carriers or excipients for the additional therapeutic agent. See, e.g., Remington's Pharmaceutical Sciences and U.S. Pharmacopeia: National Formulary, Mack Publishing Company, Easton, PA (1984); Hardman et al. (2001) Goodman and Gilman's The Pharmacological Basis of Therapeutics. McGraw-Hill, New York, NY; Gennaro (2000) Remington: The Science and Practice of Pharmacy, Lippincott, Williams, and Wilkins, New York, NY; Avis et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY: Weiner and Kotkoskie (2000) Excipient Toxicity and Safety, Marcel Dekker, Inc., New York, NY.

In some embodiments, the additional therapeutic agent is administered by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

In certain embodiments, dosing will comprise administering to a subject, escalating doses of 1.0, 3.0, and 10 mg/kg of the additional therapeutic agent, over the course of treatment. The formulation can be a reconstituted liquid formulation, or it can be a liquid formulation not previously lyophilized. Time courses can vary and can continue as long as desired effects are obtained. In certain embodiments, dose escalation will continue up to a dose of about 10 mg/kg. In certain embodiments, the subject will have a histological or cytological diagnosis of melanoma, or other form of solid tumor, and in certain instances, a subject may have non-measurable disease. In certain embodiments, the subject will have been treated with other chemotherapeutics, while in other embodiments, the subject will be treatment naïve.

In certain embodiments, the dosing regimen will comprise administering a dose of from about 0.005 mg/kg to about 10 mg/kg of an additional therapeutic agent, with intra-patient dose escalation. In certain embodiments, a dose of 5 mg/kg or 10 mg/kg will be administered at intervals of every 3 weeks, or every 2 weeks. In yet additional embodiments, a dose of 3 mg/kg will be administered at three-week intervals for melanoma patients or patients with other solid tumors. In these embodiments, patients should have non-resectable disease; however, patients may have had previous surgery.

Subcutaneous administration may be performed by injection using a syringe, or using other injection devices (e.g., the Inject-ease® device); injector pens; or needleless devices (e.g., MediJector and BioJector®).

Embodiments of the invention also include one or more of the reconstituted lyosphere formulations described herein (i) for use in, (ii) for use as a medicament or pharmaceutical formulation for, or (iii) for use in the preparation of a medicament for: (a) therapy (e.g., of the human body); (b) medicine; (c) induction of or increasing of an antitumor immune response (d) decreasing the number of one or more tumor markers in a patient; (e) halting or delaying the growth of a tumor or a blood cancer; (f) halting or delaying the progression of PD-1-related disease: (g) halting or delaying the progression cancer; (h) stabilization of PD-1-related disease: (i) inhibiting the growth or survival of tumor cells; (j) eliminating or reducing the size of one or more cancerous lesions or tumors; (k) reduction of the progression, onset or severity of PD-1-related disease: (1) reducing the severity or duration of the clinical symptoms of PD-1-related disease such as cancer (m) prolonging the survival of a patient relative to the expected survival in a similar untreated patient n) inducing complete or partial remission of a cancerous condition or other PD-1 related disease, or o) treatment of cancer.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1. John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research. St. Louis, MO: pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor. NY, pp. 139-243; Carpenter, et al. (2000) J. Immunol. 165:6205; He, et al. (1998) J. Immunol. 160:1029; Tang et al. (1999) J. Biol. Chem. 274:27371-27378: Baca et al. (1997) J. Biol. Chem. 272:10678-10684: Chothia et al. (1989) Nature 342:877-883; Foote and Winter (1992) J. Mol. Biol. 224:487-499; U.S. Pat. No. 6,329,511).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ: Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY: Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax. Inc. Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742: Wren, et al. (2002) Comput. Methods Programs Biomed. 68:177-181: von Heijne (1983) Eur. J. Biochem. 133:17-21: von Heijne (1986) Nucleic Acids Res. 14:4683-4690).

### Analytical Methods

Analytical methods suitable for evaluating the product stability include size exclusion chromatography (SEC), dynamic light scattering test (DLS), differential scanning calorimetery (DSC), iso-asp quantification, potency, UV at 340 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the alpha-carboxyl position, generating S-adenosyl-L-homocysteine (SAH) in the process. This is a relatively small molecule and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

All publications mentioned herein are incorporated by reference for the purpose of describing and disclosing methodologies and materials that might be used in connection with the invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### Example 1

### Materials and Methods

CE-SDS: Samples were analyzed by a CE-SDS technique in which protein was denatured with sodium dodecyl sulfate (SDS) under reducing and non-reducing conditions and separated using capillary electrophoresis (CE) (Beckman-Coulter ProteomeLab PA800 CE system and IgG Purity/Heterogeneity Assay Kit). The method separates proteins based on their apparent molecular weight. Under non-reducing conditions, all species other than the main IgG peak were classified as impurities. Under reducing conditions, the IgG was resolved into the heavy and light chains. All other species were classified as impurities. In both cases, the result was reported as corrected area percent of each peak as calculated from the total corrected peak area percent.
HP-IEX: High performance ion-exchange chromatography (HP-IEX) was used to assess the charge profile. An ion exchange HPLC method was performed using a Dionex ProPac WCX-10 column and a UV detector at 280 nm. Samples were diluted in purified water, and 80 μg were injected for analysis. The mobile phase used for the IEX analysis was a gradient of the following mobile phases (mobile phase A: 24 mM MES, pH 6, 4% acetonitrile (v/v); mobile phase B: 20 mM phosphate, 95 mM NaCl, pH 8, 4% acetonitrile (v/v). The main peak is the major component of the chromatogram and it serves as a control for the characterization of acidic and basic variants. Acidic variants elute earlier than main peak and the main cause of the formation of acidic variants is due to the deamidation of the Asn in main peak and the presence of sialic acid compared to main peak. Basic variants elute later than main peak and the main cause of the formation of basic variants is due to the incomplete removal of C-terminal Lys from the main peak. Other causes are incomplete cyclization of the N-terminal glutamine (Gln) to pyroGlu of the light chain or heavy chain or both and also due to the Isomerization of Asp in the main peak to isoAsp.
HP-SEC: Purity of the sample was assessed by size exclusion chromatography (SEC) in which the percentage of monomer was determined, as well as the percentages of high molecular weight species (HMW) and late eluting peaks (LMW species). The presence of HMW species indicates protein aggregates and the presence of LMW species indicate protein fragments. High Performance-Size Exclusion Chromatography (HP-SEC) was performed by diluting the samples to 1.0 mg/mL with water. The diluted samples were injected (10 μL) into a HPLC equipped with a YMC-pack-Diol 200 column and a UV detector. Proteins in the sample were separated by size and detected by UV absorption at 280 nm.
HP-SEC Arg: Purity of the sample was assessed by size exclusion chromatography (SEC) in which the percentage of monomer was determined, as well as the percentages of high molecular weight species (HMW) and late eluting peaks (LMW species). High Performance-Size Exclusion Chromatography (HP-SEC) was performed by diluting the samples to 5.0 mg/mL in mobile phase (50 mM sodium phosphate, 450 mM arginine mono hydrochloride, pH 7.0). The column temperature was set at 25° C., and the flow rate was maintained at 0.5 mL/min using an isocratic elution. The diluted samples were injected (30 μL) into a HPLC equipped with YMC-PACK Diol-200 column and a UV detector. Proteins in the sample were separated by size and detected by UV absorption at 280 nm.
A350: UV absorption at 350 nm was measured using 96 well plate Spectramax reader as an indication of turbidity. The absorption readings were blanked against empty plate reading and normalized for sample pathlength.
HP-HIC: High performance hydrophobic interaction chromatography (HP-HIC) was used to assess oxidized products from the non-oxidized molecule. The percentage of pre-peaks, determined to be oxidized species comprising heavy chain Met105 oxidation on one heavy chain by previous analytical characterization, as well as the percentage of the main and percentage of the post peaks were determined. A HP-HIC method was performed by diluting the sample to 5.0 mg/mL in purified water. The sample was then injected (10 μL) into an HPLC equipped with a Tosoh Phenyl-5PW column and a UV detector at 280 nm. For the HIC analysis a mobile phase containing a gradient of the following components (mobile phase A: 5 mM sodium phosphate in 2% acetonitrile, pH 7.0; mobile phase B: 400 mM ammonium sulfate, 5 mM sodium phosphate in 2% acetonitrile, pH 6.9;) was used.
VP-DSC: Valerian-Plotnikov differential scanning calorimetry (VP-DSC) can be used to determine the thermal and conformational stability of monoclonal antibodies. DSC determines the heat capacity (Cp) of the protein solution relative to that of the placebo solution for increasing temperatures, producing a thermal transition upon protein unfolding. For monoclonal antibodies, multiple unfolding transitions (Tonset, Tml, Tm2) are typically seen in the DSC thermogram corresponding to unfolding of individual domains

### Example 2

### Manufacture of Lyospheres:

Lyosphere frozen beads were manufactured by dispensing 50 μL increments of the formulations of the instant invention onto a −180° C. aluminum plate and collecting in a pre-chilled glass bottle. Each formulation bottle was placed at −70° C. until lyophilization.

The lyospheres were dried by lyophilization in either a LyoStar2 or a LyoStar3 instrument. The frozen lyosphere beads were poured into a single layer that had been pre-cooled on the −50° C. shelf in the lyophilizer. The cycle included freezing at −50° C. for 30 minutes, then apply vacuum to 30 mTorr and hold for 60 minutes at −50° C. The shelf temperature was ramped to 15° C. at 0.1° C./min and held at 15° C. for 1440 minutes. The shelf temperature was ramped from 15° C. to 30° C. at 0.1° C./min and held for 360 minutes. Lastly, the shelf temperature was set to −50° C. until unloaded. The unloaded process was performed under low relative humidity (<4%) in a nitrogen glove box. Dried beads were transferred from the lyophilization trays to glass bottles and capped. The bottles were then placed in aluminum foil bags and heat sealed until dispensed.

### Example 3

### Evaluation of Anti-PD-1 Antibody by Lyophilization and Microwave Vacuum Drying

Pembrolizumab was used in this experiment. Methods for making pembrolizumab are described in WO2008/156712.

Pembrolizumab at a concentration of 50 mg/mL was dialyzed into two formulations: (1) 7% sucrose, 10 mM histidine, pH 6.0 (F1) and (2) 12.5% sucrose, 12.5% trehalose, 10 mM histidine, pH 6.0 (F2). After dialysis, the formulations were filtered through an 0.22 μm filter unit and then PS-80 surfactant was added to a final concentration of 0.05% (see Table 3).

TABLE 3
Pembrolizumab Formulations

| Identifier | Formulation Composition |
|---|---|
| F1 | 50 mg/mL pembrolizumab, 7% sucrose, 10 mM Histidine, |
| | 0.05% PS-80, pH 6.0 |
| F2 | 50 mg/mL pembrolizumab, 12.5% sucrose, 12.5% trehalose, |
| | 10 mM Histidine, 0.05 PS-80, pH 6.0 |

The F1 and F2 formulations of pembrolizumab were filled into vials. Containers were frozen at −115° C. for 15 minutes. The F1 and F2 formulation vials were dried via lyophilization cycles. The annealing step was performed by cycling from −50° C. to −10° C. two times. The primary drying cycle was performed at −35° C. for 10,800 minutes at 54 mTorr. The secondary drying cycle was performed at 5° C. for 600 minutes at 54 mTorr.

Lyophilized vials were placed at 40° C., and vials were pulled at 1, 3 and 6 months. Moisture content of the dried samples was measured.

The lyophilized samples were reconstituted with 450 μL of sterile filtered water to 100 mg/mL pembrolizumab. The samples were diluted to a concentration of 1 mg/mL in water. The samples underwent size exclusion (SEC) analysis. A volume (200 L) of each sample was aliquoted into HPLC vials and 10 μL of each sample was injected in duplicate onto a SEC column. The method utilized was an isocratic run for 30 minutes at 0.5 mL/min in a mobile phase (50 mM phosphate, 200 mM NaCl, pH 7.0). The amount of high molecular weight species was quantitated and plotted for each time point.

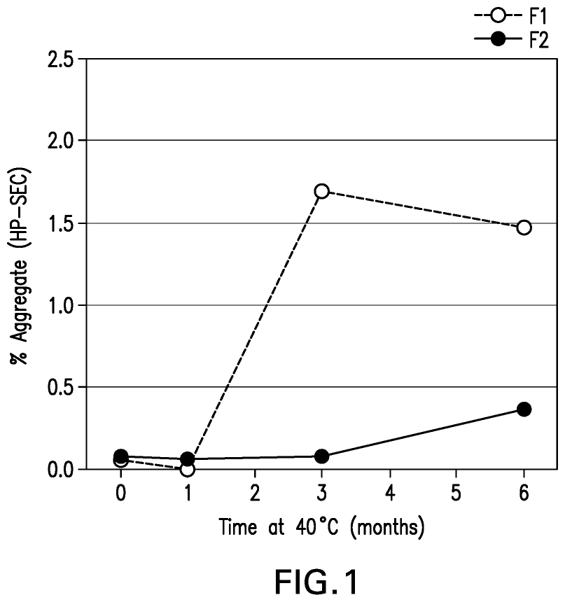

Data show that the anti-PD-1 antibody, pembrolizumab, was stable up to 6 months at 40° C. in the F2 formulation. The data in Example 3 demonstrate that the high disaccharide to anti-PD-1 antibody ratio (ratio=5 in F2) resulted in less aggregation than the anti-PD-1 antibody formulation with lower disaccharide to anti-PD-1 antibody ratio (ratio=1.4 in F1) (FIG. 1).

The reconstitution time was approximately 6 minutes and the moisture content was less than 5%. Table 4 shows the residual moisture percentage calculated for the pembrolizumab formulations.

TABLE 4
Moisture data

| | | Residual Moisture (%) |
|---|---|---|
| F1 | Lyophilized | 4.35 |
| F2 | Lyophilized | 4.20 |

### Example 4

### Anti-PD-1 Antibody Formulations

The anti-PD-1 antibody (pembrolizumab) was separately formulated in the presence of 0.2 mg/mL PS-80 in 10 mM histidine buffer at pH 5.5 with pembrolizumab concentrations ranging from 25 to 150 mg/mL. Sucrose, trehalose or a combination of both was used in the formulations at concentrations from 70 to 220 mg/mL (Table 5). This study demonstrated manufacture feasibility to dry pembrolizumab formulations to lyosphere beads and ability to achieve concentrations from 25 to 200 mg/mL after reconstitution without impact to product quality attributes after drying and reconstitution.

### Manufacture of Lyospheres:

Lyosphere frozen beads (lyospheres) were manufactured by dispensing 50 μL increments of pre-lyosphere formulations onto a −180° C. aluminum plate and collecting in a pre-chilled glass bottle. Each formulation bottle was placed at −70° C. until lyophilization.

The lyospheres were dried by lyophilization in either a LyoStar2 or a LyoStar3 instrument (a lyophilizer). The frozen lyosphere beads were poured into a single layer that had been pre-cooled on the −50° C. shelf in the lyophilizer. The lyophilization cycle included freezing at −50° C. for 30 minutes, then applying vacuum (30 mTorr), and holding the samples for 60 minutes at −50° C. The shelf temperature was ramped to 15° C. at 0.1° C./min and held at 15° C. for 1440 minutes. The shelf temperature was adjusted from 15° C. to 30° C. at 0.1° C./min and held for 360 minutes. Lastly, the shelf temperature was set to −50° C. until the beads (lyospheres) were unloaded. The unloading process was performed under low relative humidity (<4%) in a nitrogen glove box. The dried lyosphere beads were transferred from the lyophilization trays to glass bottles, which were then capped. The bottles were then placed in aluminum foil bags and heat sealed until dispensed.

### Stability Testing

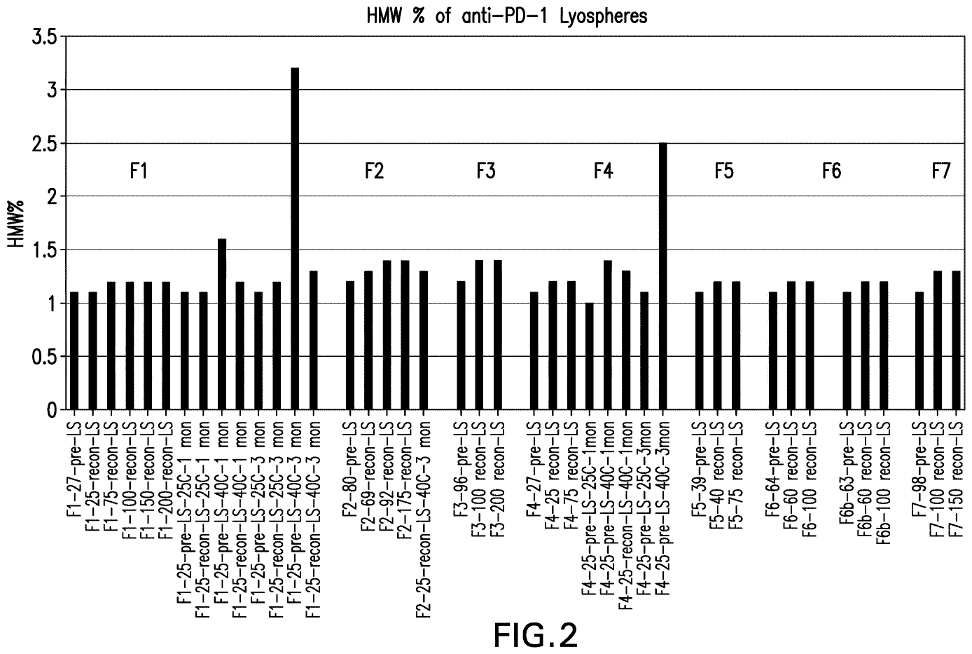

Solid lyosphere samples (lyospheres) were dispensed into 20R vials to reach various doses of anti-PD-1 antibody (pembrolizumab: 25 mg to 250 mg) under relative humidity (<10%) in a nitrogen glove box. The lyospheres were sealed and staged at 25° C./60% RH, 40° C./75% RH stability chambers. At each designated time point, the lyospheres were pulled, reconstituted to 25-250 mg/mL with a Water-For-Injection system. The level of higher order aggregation in the reconstituted samples was analyzed using Ultra Performance Size Exclusion Chromatography (UPSEC). See FIG. 2. Various novel excipients were used in the diluent to evaluate their effects on reducing reconstitution time.

TABLE 5
Anti-PD-1 Formulations

| | | Antibody | | | | | | |
|---|---|---|---|---|---|---|---|---|
| | | Conc. | Histidine | Sucrose | Trehalose | PS-80 | Arginine | |
| Form. | Antibody | (mg/ml) | (mM) | (mg/mL) | (mg/mL) | (% w/v) | (mg/mL) | pH |
| F1 | Anti-PD-1 | 25 | 10 | 70 | — | 0.02 | — | 5.5 |
| F2 | Anti-PD-1 | 75 | 10 | 70 | — | 0.02 | — | 5.5 |
| F3 | Anti-PD-1 | 150 | 10 | 70 | — | 0.02 | — | 5.5 |
| F4 | Anti-PD-1 | 25 | 10 | 70 | 150 | 0.02 | — | 5.5 |
| F5 | Anti-PD-1 | 75 | 10 | 70 | 150 | 0.02 | — | 5.5 |
| F6a | Anti-PD-1 | 100 | 10 | 70 | 80 | 0.02 | — | 5.5 |
| F6b | Anti-PD-1 | 100 | 10 | — | 150 | 0.02 | — | 5.5 |
| F7 | Anti-PD-1 | 150 | 10 | 70 | 80 | 0.02 | — | 5.5 |

### Results

All lyosphere formulations were observed to be stable after reconstitution to various concentration levels (25 to 200 mg/mL). For F1 and F4, the formulations were stable for 3-months when stored at 40° C. with 75% relative humidity with less than 1.5% aggregation after reconstitution to 25 mg/mL (FIG. 2).

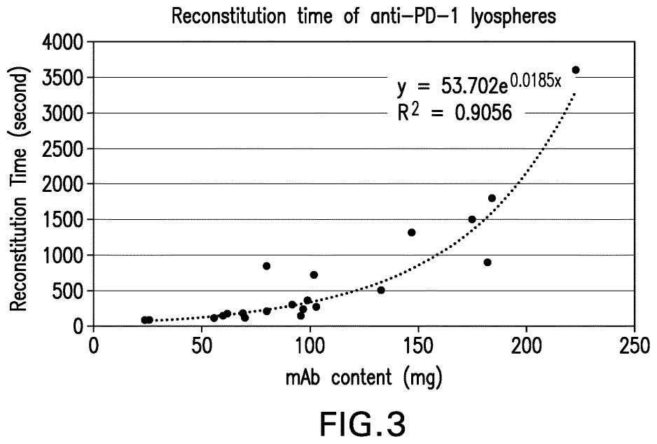

The data demonstrated the viscosity of the anti-PD-1 mAb formulation as the dominating factor for reconstitution time (FIG. 3).

### Example 5

### Anti-PD-I Antibody Formulation Stability Analysis

An anti-PD-1 antibody (pembrolizumab) was used in this experiment. Methods for making this anti-PD-1 antibody are described in WO2008/156712.

The anti-PD-1 antibody (pembrolizumab) was formulated at a fixed concentration of 100 mg/mL in 10 mM histidine buffer at pH 5.5 with either 100 mg/mL sucrose, or a 10 combination of 25 mg/mL sucrose with 75 mg/mL mannitol. The ratio of disaccharide (sucrose or a combination of sucrose and mannitol) to pembrolizumab was 1:1. Lyospheres were generated and were reconstituted to achieve concentrations double the concentrations in the pre-lyosphere compositions. Three anti-PD-1 antibody formulations were designed (Table 6) 1:1 w:w ratio with stabilizer to evaluate reconstitution time impacted by the type of excipient in the formulation or the type of diluent used and long term physical and chemical stability at 12 months. The anti-PD-1 antibody formulations were prepared as lyospheres and lyophilized cakes as described above. The reconstituted formulations F1, F2 and F3 stored at 12 months showed better stability when stored at 25° C./60% RH than 40° C./75% RH.

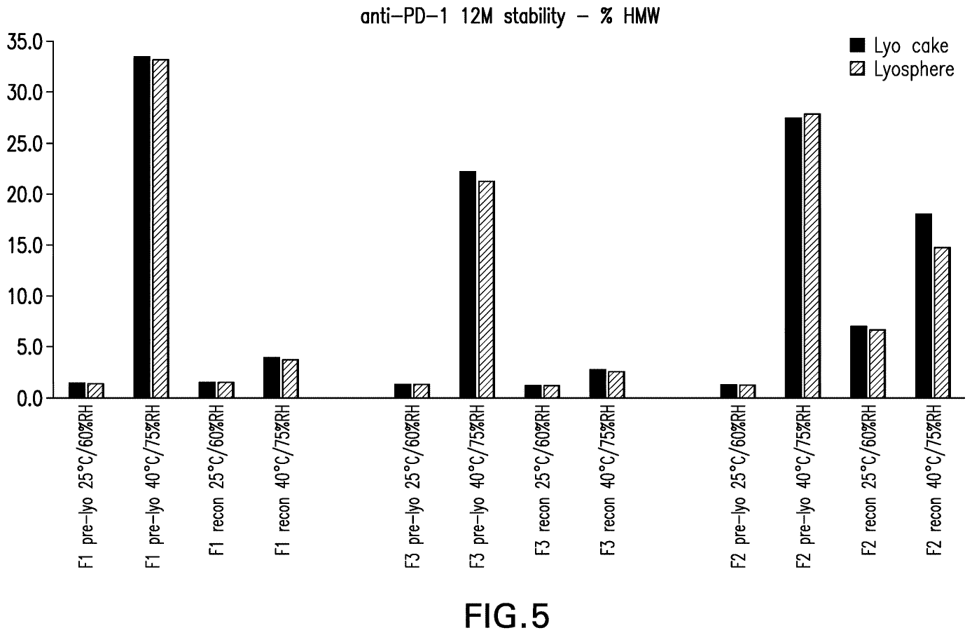

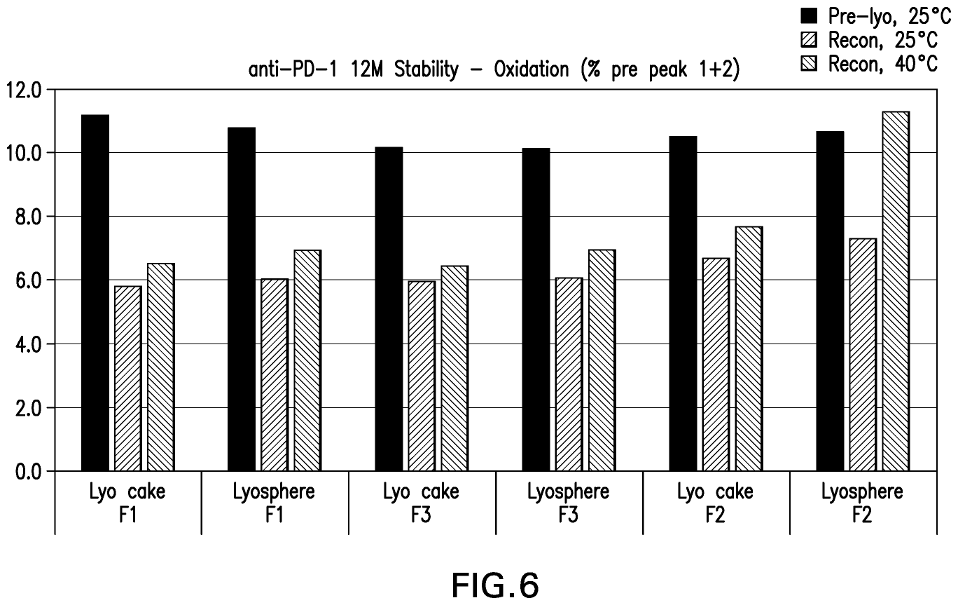

Data show that after 12 months storage at 25° C./60% RH or at 40° C./75% RH, the lyospheres and lyophilized cakes prepared with anti-PD-1 F1 antibody formulation and the lyospheres and lyophilized cakes prepared with anti-PD-1 F3 antibody formulation showed comparable physical and chemical stability post reconstitution. Percent HMW species were all below 5% (FIG. 5). Oxidation species were below 10% (FIG. 6). The lyospheres and lyophilized cakes prepared with the F2 anti-PD-1 antibody formulation showed more degradation compared to the lyospheres and lyophilized cakes prepared with the anti-PD-1 F1 antibody formulation and the lyospheres and lyophilized cakes prepared with the anti-PD-1 F3 antibody formulation due to phase separation of stabilizer (mannitol) from the amorphous mAb during the freeze-drying process. The data demonstrate potential of room temperature stability of anti-PD-1 formulated with sucrose at 1:1 weight ratio (F1).

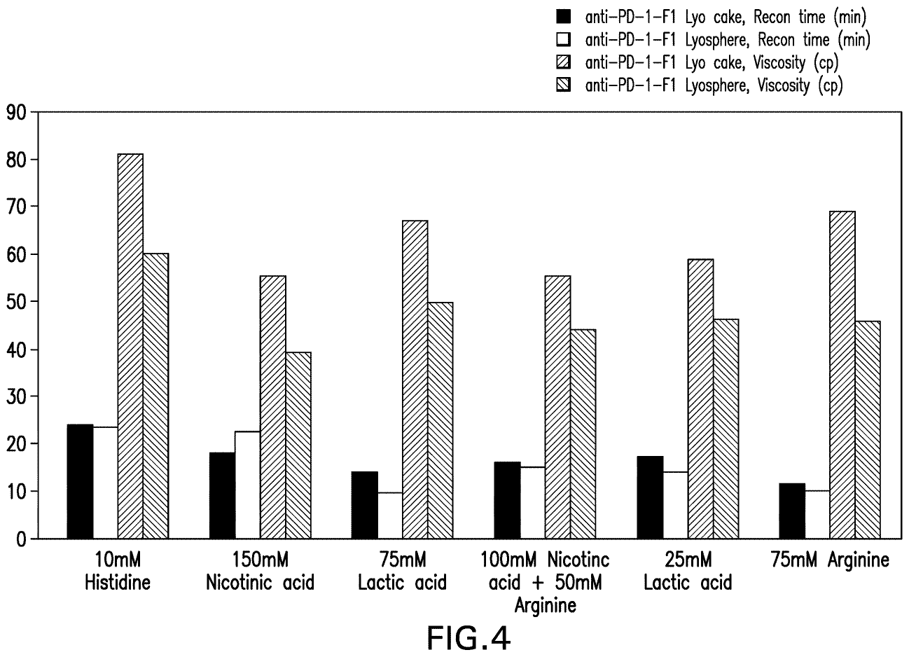

An additional study was performed to test the utility of alternative excipients as viscosity lowering agents in high concentration pembrolizumab formulations. Data show that viscosity-reducing excipients reduced reconstitution time. Lactic acid showed reduction in reconstitution time (FIG. 4).

TABLE 6
Formulation composition of anti-PD-1 at 100 mg/mL prior to drying process

| | | Concentration | | | | |
|---|---|---|---|---|---|---|
| | | of mAb pre-lyo | PS80 | | | |
| mAb | Formulation | (mg/mL) | (mg/mL) | Disaccharide | Buffer | pH |
| PD-1 | F1 | 100 | 0.2 | 100 mg/mL sucrose | 10 mM | 5.5 |
| | | | | | histidine | |
| PD-1 | F2 | 100 | 0.2 | 25 mg/mL sucrose + | 10 mM | 5.5 |
| | | | | 75 mg/mL mannitol | histidine | |
| PD-1 | F3 | 100 | 0.2 | 100 mg/mL sucrose | 10 mM | 5.5 |
| | | | | | histidine + | |
| | | | | | 75 mM | |
| | | | | | Arginine | |

Note:
the dried formulations were reconstituted to 50% of the initial fill volume to achieve ~200 mg/mL post reconstitution.

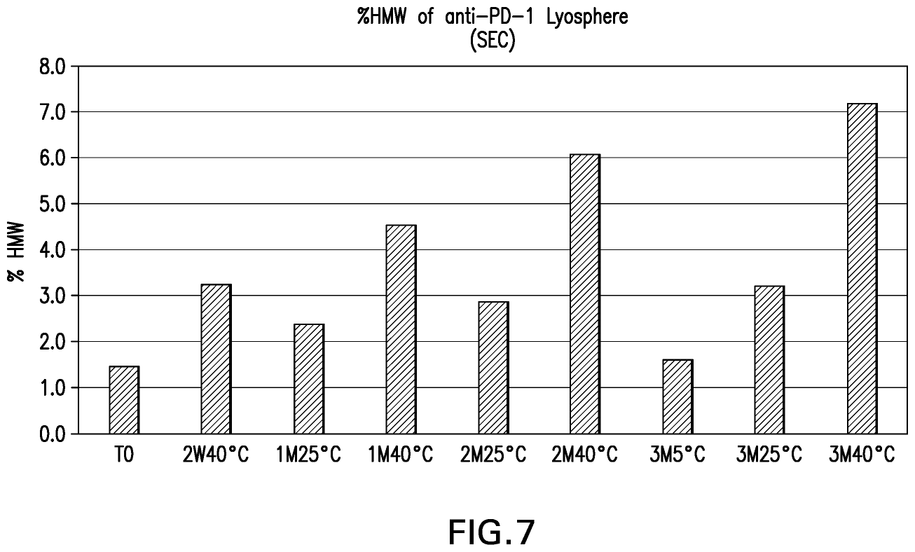

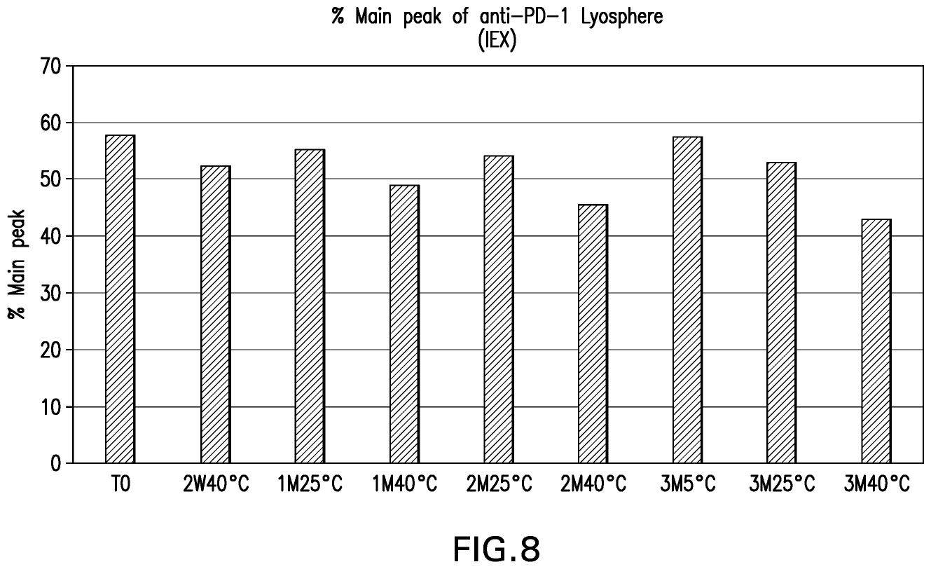

In a follow-up study, the anti-PD-1 antibody (pembrolizumab) was formulated at a 5 concentration of 165 mg/mL with 70 mg/mL of sucrose, 1.47 mg/mL L-methionine and 0.2 mg/mL PS-80 in 10 mM histidine buffer at pH 5.5. The ratio of disaccharide (sucrose) to pembrolizumab was 0.42:1. Lyospheres were generated and were reconstituted to achieve the same concentrations as the in pre-lyosphere composition. This study aimed to assess stability of pembrolizumab formulated at 165 mg/mL with a lower weight ratio of stabilizer to pembrolizumab (0.42:1). Available SEC and IEX data on the formulation containing 165 mg/mL antibody and 70 mg/mL sucrose showed an increase in aggregation or loss of main peak over time at storage conditions of 25° C., and 40° C. The data suggest the ratio of disaccharide to antibody (0.42:1) is too low, and a higher disaccharide to antibody ratio is required for optimal stabilization during the freezing and drying process (FIG. 7 and FIG. 8).

1. A lyosphere comprising an anti-PD-1 antibody and a disaccharide wherein the concentration of disaccharide is equal to or greater than the concentration of anti-PD-1 antibody.

2. The lyosphere of claim 1 further comprising a surfactant.

3. The lyosphere of claim 1 wherein the anti-PD-1 antibody concentration is at least 1.25 mg and the disaccharide concentration is at least 1.25 mg.

4. The lyosphere of claim 2 wherein the surfactant concentration is 0.005 to 0.01 mg.

5. The lyosphere of claim 1 wherein the anti-PD-1 antibody is pembrolizumab or a variant thereof.

6. The lyosphere of claim 1 any of claims 1-4 wherein the anti-PD-1 antibody is nivolumab or a variant thereof.

7. The lyosphere of claim 1 wherein the anti-PD-1 antibody is at a concentration of about 1.25 mg, or about 2 mg, or about 4 mg, or about 6 mg, or about 8 mg, or about 10 mg.

8. The lyosphere of claim 1 wherein the disaccharide is at a concentration of about 1.25 mg, or about 2 mg, or about 4 mg, or about 6 mg, or about 8 mg, or about 10 mg, or about 12 mg, or about 12.5 mg.

9. The lyosphere of claim 1 wherein the disaccharide is sucrose or trehalose, or a combination of sucrose and trehalose.

10. The lyosphere of claim 1 wherein the disaccharide is a combination of sucrose and trehalose, wherein the concentration of trehalose is greater than concentration of sucrose.

11. The lyosphere of claim 2 wherein the surfactant is a non-ionic surfactant.

12. The lyosphere of claim 11 wherein the surfactant is polysorbate 80 (PS-80) or polysorbate 20 (PS-20).

13. The lyosphere of claim 12 wherein the surfactant is PS-80.

14. The lyosphere of claim 12 wherein the surfactant is PS-20.

15. The lyosphere of claim 2 wherein the surfactant is at a concentration of about 0.05 to 0.1 mg.

16. The lyosphere of claim 15 wherein the surfactant is at a concentration of about 0.5 to 1 mg.

17. The lyosphere of claim 1 wherein the ratio of anti-PD-1 antibody to disaccharide is about 1:1, or about 1:1.5, or about 1:2, or about 1:2.5, or about 1:3.

18. The lyosphere of claim 1 wherein the lyosphere is reconstituted.

19. A reconstituted lyosphere formulation comprising: a) 130 mg/mL to 200 mg/mL of an anti-PD-1 antibody; b) 130 mg/mL to 200 mg/mL disaccharide; c) 1 to 20 mM buffer, pH 5.0-6.5; and d) about 0.02-0.04% surfactant.

20. A reconstituted lyosphere formulation comprising: a) 130 mg/mL to 200 mg/mL of pembrolizumab; b) 130 mg/mL to 200 mg/mL trehalose, or 130 mg/mL to 200 mg/mL sucrose, or a combination of trehalose and sucrose totaling 130 mg/mL to 200 mg/mL; c) 10 mM histidine buffer, pH 5.5-6.0; and d) 0.02-0.04% PS-80.

21. The reconstituted lyosphere formulation according to claim 19 useful for subcutaneous administration to a patient for the treatment of a disease or condition.

22. The reconstituted lyosphere formulation according to claim 19 useful for subcutaneous administration to a patient for the treatment of cancer.

23. A method of treating a patient with cancer, comprising administering to the patient a reconstituted lyosphere formulation of claim 19.

24. The method of claim 23, wherein the formulation is administered to the patient intravenously or subcutaneously.

25. The method of claim 23, wherein the cancer is selected from the group consisting of: melanoma, non-small cell lung cancer, small cell lung cancer, head and neck cancer, urothelial cancer, breast cancer, gastrointestinal cancer, gastroesophageal junction adenocarcinoma, multiple myeloma, hepatocellular cancer, non-Hodgkin lymphoma, primary mediastinal large B-cell lymphoma, renal cancer, Hodgkin lymphoma, mesothelioma, ovarian cancer, esophageal cancer, anal cancer, biliary tract cancer, colorectal cancer, cervical cancer, endometrial cancer, cutaneous squamous cell cancer, thyroid cancer, prostate cancer, glioblastoma, Merkel cell carcinoma, and salivary cancer.

26. The method of claim 23, wherein the patient has a microsatellite instability-high or mismatch repair deficient solid tumor.

27. The method of claim 23, wherein the patient has a tumor with a high mutational burden.


SEQUENCE LISTING (20 sequences)

SEQ ID NO: 1 - protein, 15 aa, synthetic construct
RASKGVSTSGYSYLH

SEQ ID NO: 2 - protein, 7 aa, synthetic construct
LASYLES

SEQ ID NO: 3 - protein, 9 aa, synthetic construct
QHSRDLPLT

SEQ ID NO: 4 - protein, 111 aa, synthetic construct [sequence omitted]

SEQ ID NO: 5 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
NYYMY

SEQ ID NO: 7 - protein, 17 aa, synthetic construct
GINPSNGGTNFNEKFKN

SEQ ID NO: 8 - protein, 11 aa, synthetic construct
RDYRFDMGFDY

SEQ ID NO: 9 - protein, 120 aa, synthetic construct [sequence omitted]

SEQ ID NO: 10 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, synthetic construct
RASQSVSSYLA

SEQ ID NO: 12 - protein, 7 aa, synthetic construct
DASNRAT

SEQ ID NO: 13 - protein, 9 aa, synthetic construct
QQSSNWPRT

SEQ ID NO: 14 - protein, 107 aa, synthetic construct [sequence omitted]

SEQ ID NO: 15 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
NSGMH

SEQ ID NO: 17 - protein, 17 aa, synthetic construct
VIWYDGSKRYYADSVKG

SEQ ID NO: 18 - protein, 4 aa, synthetic construct
NDDY

SEQ ID NO: 19 - protein, 113 aa, synthetic construct [sequence omitted]

SEQ ID NO: 20 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK